SEQUENCE LISTING (30 sequences)

SEQ ID NO: 1 - DNA, 186 bp, Homo sapiens
atgctgcccggtttggcactgctcctgctggccgcctggacggctcgggcgcttgatgcagaattccgacatgactcaggatatgaagttcatcatcaaaaattggtgttctttgcagaagatgtgggttcaaacaaaggtgcaatcattggactcatggtgggcggtgttgtcatagcgacttaa

SEQ ID NO: 2 - DNA, 537 bp, Mus musculus
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 3 - DNA, 537 bp, Homo sapiens
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 4 - DNA, 14412 bp, Artificial
  a DNA endoding a Sendai virus antigenomic RNA
accaaacaagagaaaaaacatgtatgggatatgtaatgaagttatacaggattttagggtcaaagtatccaccctgaggagcaggttccagaccctttgctttgctgccaaagttcacgcggccgccaaggttcacttatgctgcccggtttggcactgctcctgctggccgcctggacggctcgggcgcttgatgcagaattccgacatgactcaggatatgaagttcatcatcaaaaattggtgttctttgcagaagatgtgggttcaaacaaaggtgcaatcattggactcatggtgggcggtgttgtcatagcgacttaaccgtagtaagaaaaacttagggtgaaagttcatcgcggccgcagatcttcacgatggccgggttgttgagcaccttcgatacatttagctctaggaggagcgaaagtattaataagtcgggaggaggtgctgttatccccggccagaggagcacagtctcagtgttcgtactaggcccaagtgtgactgatgatgcagacaagttattcattgcaactaccttcctagctcactcattggacacagataagcagcactctcagagaggggggttcctcgtctctctgcttgccatggcttacagtagtccagaattgtacttgacaacaaacggagtaaacgccgatgtcaaatatgtgatctacaacatagagaaagaccctaagaggacgaagacagacggattcattgtgaagacgagagatatggaatatgagaggaccacagaatggctgtttggacctatggtcaacaagagcccactcttccagggtcaacgggatgctgcagaccctgacacactccttcaaatctatgggtatcctgcatgcctaggagcaataattgtccaagtctggattgtgctggtgaaggccatcacaagcagcgccggcttaaggaaagggttcttcaacaggttagaggcgttcagacaagacggcaccgtgaaaggtgccttagttttcactggggagacagttgaggggataggctcggttatgagatctcagcaaagccttgtatctctcatggttgagacccttgtgactatgaatactgcaagatctgatctcaccacattagagaagaacatccagatcgttgggaactacatccgagatgcagggctggcttccttcatgaacactattaaatatggggtggaaacaaagatggcagctctaacgttgtcaaacctgaggcccgatattaataagcttagaagcctcatagacacctacctgtcaaaaggccccagagctccctttatctgtatcctcaaggaccctgttcatggtgaatttgctccaggcaattatcctgcactatggagttacgccatgggagtcgccgtcgtacagaacaaggcaatgcagcagtacgtcacagggaggacataccttgatatggaaatgttcttactaggacaagccgtggcaaaggatgctgaatcgaagatcagcagtgccttggaagatgagttaggagtgacggatacagccaaggggaggctcagacatcatctggcaaacttgtccggtggggatggtgcttaccacaaaccaacaggcggtggtgcaattgaggtagctctagacaatgccgacatcgacctagaaacaaaagcccatgcggaccaggacgctaggggttggggtggagatagtggtgaaagatgggcacgtcaggtgagtggtggccactttgtcacactacatggggctgaacggttagaggaggaaaccaatgatgaggatgtatcagacatagagagaagaatagccatgagactcgcagagagacggcaagaggattctgcaacccatggagatgaaggccgcaataacggtgtcgatcatgacgaagatgacgatgccgcagcagtagctgggataggaggaatctaggatcatacgaggcttcaaggtacttgatccgtagtaagaaaaacttagggtgaaagttcatccaccgatcggctcaggcaaggccacacccaaccccaccgaccacacccagcagtcgagacagccacggcttcggctacacttaccgcatggatcaagatgccttcattcttaaagaagattctgaagttgagagggaggcgccaggaggacgagagtcgctctcggatgttatcggattcctcgatgctgtcctgtcgagtgaaccaactgacatcggaggggacagaagctggctccacaacaccatcaacactccccaaggaccaggctctgctcatagagccaaaagtgagggcgaaggagaagtctcaacaccgtcgacccaagataatcgatcaggtgaggagagtagagtctctgggagaacaagcaagccagaggcagaagcacatgctggaaaccttgataaacaaaatatacaccgggcctttgggggaagaactggtacaaactctgtatctcaggatctgggcgatggaggagactccggaatccttgaaaatcctccaaatgagagaggatatccgagatcaggtattgaagatgaaaacagagagatggctgcgcaccctgataagaggggagaagaccaagctgaaggacttccagaagaggtacgaggaagtacatccctacctgatgaaggagaaggtggagcaagtaataatggaagaagcatggagcctggcagctcacatagtgcaagagtaactggggtcctggtgattcctagccccgaacttgaagaggctgtgctacggaggaacaaaagaagacctaccaacagtgggtccaaacctcttactccagcaaccgtgcctggcacccggtccccaccgctgaatcgttacaacagcacagggtcaccaccaggaaaacccccatctacacaggatgagcacatcaactctggggacacccccgccgtcagggtcaaagaccggaaaccaccaatagggacccgctctgtctcagattgtccagccaacggccgcccaatccacccgggtctagagaccgactcaacaaaaaagggcataggagagaacacatcatctatgaaagagatggctacattgttgacgagtcttggtgtaatccagtctgctcaagaattcgaatcatcccgagacgcgagttatgtgtttgcaagacgtgccctaaagtctgcaaactatgcagagatgacattcaatgtatgcggcctgatcctttctgccgagaaatcttccgctcgtaaggtagatgagaacaaacaactgctcaaacagatccaagagagcgtggaatcattccgggatatttacaagagattctctgagtatcagaaagaacagaactcattgctgatgtccaacctatctacacttcatatcatcacagatagaggtggcaagactgacaacacagactcccttacaaggtccccctccgtttttgcaaaatcaaaagagaacaagactaaggctaccaggtttgacccatctatggagaccctagaagatatgaagtacaaaccggacctaatccgagaggatgaatttagagatgagatccgcaacccggtgtaccaagagagggacacagaacccagggcctcaaacgcatcacgtctctttccctccaaagagaagcccacaatgcactctctcaggctcgtcatagagagcagtcccctaagcagagctgagaaagtagcatatgtgaaatcattatccaagtgcaagacagaccaagaggttaaggcagtcatggaactcgtagaagaggacatagagtcactgaccaactagatcccgggtgaggcatcctaccatcctcagtcatagagagatccaatctaccatcagcatcagccagtaaagattaagaaaaacttagggtgaaagaaatttcacctaacacggcgcaatggcagatatctatagattccctaagttctcatatgaggataacggtactgtggagcccctgcctctgagaactggtccggataagaaagccatcccccacatcaggattgtcaaggtaggagaccctcctaaacatggagtgagatacctagatttattgctcttgggtttctttgagacaccgaaacaaacaaccaatctagagagcgtatctgacttgacagagccgaccagctactcaatatgcggctccgggtcgttacccataggtgtggccaaatactacgggactgatcaggaactcttaaaggcctgcaccgatctcagaattacggtgaggagggctgttcgagcaggagagatgatcgtatacatggtggattcgattggtgctccactcctaccatggtcaggcaggctgagacagggaatgatatttaatgcaaacaaggtcgcactagctccccaatgcctccctgtggacaaggacataagactcagagtggtgtttgtcaatgggacatctctaggggcaatcaccatatccaagatcccaaagacccttgcagaccttgcattgcccaactctatatctgttaatttactggtgacactcaagaccgggatctccacagaacaaaagggggtactcccagtacttgatgatcaaggggagaaaaagctcaattttatggtgcacctcgggttgatcaggagaaaggtcgggaagatatactctgttgagtactgcaagagcaagattgagagaatgcggctgattttctcacttgggttaatcggcggtataagcttccatgttcaggttaatgggacactatctaagacattcatgagtcagctcgcatggaagagggcagtctgcttcccattaatggatgtgaatccccatatgaacatggtgatttgggcggcatctgtagaaatcacaggcgtcgatgcggtgttccaaccggccatccctcgtgatttccgctactaccctaatgttgtggctaagaacatcggaaggatcagaaagctgtaaatgtgcacccatcagagacctgcgacaatgccccaagcagacaccacctggcagtcggagccaccgggtcactccttgtcttaaataagaaaaacttagggataaagtcccttgtgagtgcttggttaattaagcggccgccaaagttcaatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaccgtagtaagaaaaacttagggtgaaagttcatcgcggccgcctcaaacaagcacagatcatggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtgacaacccggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaatgaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaaatttaactgactagcaggcttgtcggccttgctgacactagagtcatctccgaacatccacaatatctctcagtctcttacgtctctcacagtattaagaaaaacccagggtgaatgggaagcttgccataggtcatggatgggcaggagtcctcccaaaacccttctgacatactctatccagaatgccacctgaactctcccatagtcagggggaagatagcacagttgcacgtcttgttagatgtgaaccagccctacagactgaaggacgacagcataataaatattacaaagcacaaaattaggaacggaggattgtccccccgtcaaattaagatcaggtctctgggtaaggctcttcaacgcacaataaaggatttagaccgatacacgtttgaaccgtacccaacctactctcaggaattacttaggcttgatataccagagatatgtgacaaaatccgatccgtcttcgcggtctcggatcggctgaccagggagttatctagtgggttccaggatctttggttgaatatcttcaagcaactaggcaatatagaaggaagagaggggtacgatccgttgcaggatatcggcaccatcccggagataactgataagtacagcaggaatagatggtataggccattcctaacttggttcagcatcaaatatgacatgcggtggatgcagaagaccagaccggggggacccctcgatacctctaattcacataacctcctagaatgcaaatcatacactctagtaacatacggagatcttgtcatgatactgaacaagttgacattgacagggtatatcctaacccctgagctggtcttgatgtattgtgatgttgtagaaggaaggtggaatatgtctgctgcagggcatctagataagaagtccattgggataacaagcaaaggtgaggaattatgggaactagtggattccctcttctcaagtcttggagaggaaatatacaatgtcatcgcactattggagcccctatcacttgctctcatacaactaaatgatcctgttatacctctacgtggggcatttatgaggcatgtgttgacagagctacagactgttttaacaagtagagacgtgtacacagatgctgaagcagacactattgtggagtcgttactcgccattttccatggaacctctattgatgagaaagcagagatcttttccttctttaggacatttggccaccccagcttagaggctgtcactgccgccgacaaggtaagggcccatatgtatgcacaaaaggcaataaagcttaagaccctatacgagtgtcatgcagttttttgcactatcatcataaatgggtatagagagaggcatggcggacagtggcccccctgtgacttccctgatcacgtgtgtctagaactaaggaacgctcaagggtccaatacggcaatctcttatgaatgtgctgtagacaactatacaagtttcataggcttcaagtttcggaagtttatagaaccacaactagatgaagatctcacaatatatatgaaagacaaagcactatcccccaggaaggaggcatgggactctgtatacccggatagtaatctgtactataaagccccagagtctgaagagacccggcggcttattgaagtgttcataaatgatgagaatttcaacccagaagaaattatcaattatgtggagtcaggagattggttgaaagacgaggagttcaacatctcgtacagtctcaaagagaaagagatcaagcaagagggtcgtctattcgcaaaaatgacttataagatgcgagccgtacaggtgctggcagagacactactggctaaaggaataggagagctattcagcgaaaatgggatggttaaaggagagatagacctacttaaaagattgactactctttctgtctcaggcgtccccaggactgattcagtgtacaataactctaaatcatcagagaagagaaacgaaggcatggaaaataagaactctggggggtactgggacgaaaagaagaggtccagacatgaattcaaggcaacagattcatcaacagacggctatgaaacgttaagttgcttcctcacaacagacctcaagaaatactgcttaaactggagatttgagagtactgcattgtttggtcagagatgcaacgagatatttggcttcaagaccttctttaactggatgcatccagtccttgaaaggtgtacaatatatgttggagatccttactgtccagtcgccgaccggatgcatcgacaactccaggatcatgcagactctggcattttcatacataatcctagggggggcatagaaggttactgccagaagctgtggaccttaatctcaatcagtgcaatccacctagcagctgtgagagtgggtgtcagggtctctgcaatggttcagggtgacaatcaagctatagccgtgacatcaagagtacctgtagctcagacttacaagcagaagaaaaatcatgtctatgaggagatcaccaaatatttcggtgctctaagacacgtcatgtttgatgtagggcacgagctaaaattgaacgagaccatcattagtagcaagatgtttgtctatagtaaaaggatatactatgatgggaagattttaccacagtgcctgaaagccttgaccaagtgtgtattctggtccgagacactggtagatgaaaacagatctgcttgttcgaacatctcaacatccatagcaaaagctatcgaaaatgggtattctcctatactaggctactgcattgcgttgtataagacctgtcagcaggtgtgcatatcactagggatgactataaatccaactatcagcccgaccgtaagagatcaatactttaagggtaagaattggctgagatgtgcagtgttgattccagcaaatgttggaggattcaactacatgtctacatctagatgctttgttagaaatattggagaccccgcagtagcagccctagctgatctcaaaagattcatcagagcggatctgttagacaagcaggtattatacagggtcatgaatcaagaacccggtgactctagttttctagattgggcttcagacccttattcgtgtaacctcccgcattctcagagtataactacgattataaagaatatcactgctagatctgtgctgcaggaatccccgaatcctctactgtctggtctcttcaccgagactagtggagaagaggatctcaacctggcctcgttccttatggaccggaaagtcatcctgccgagagtggctcatgagatcctgggtaattccttaactggagttagggaggcgattgcagggatgcttgatacgaccaagtctctagtgagagccagcgttaggaaaggaggattatcatatgggatattgaggaggcttgtcaattatgatctattgcagtacgagacactgactagaactctcaggaaaccggtgaaagacaacatcgaatatgagtatatgtgttcagttgagctagctgtcggtctaaggcagaaaatgtggatccacctgacttacgggagacccatacatgggctagaaacaccagaccctttagagctcttgaggggaatatttatcgaaggttcagaggtgtgcaagctttgcaggtctgaaggagcagaccccatctatacatggttctatcttcctgactctatagacctggacacgcttacaaacggatgtccggctataagaatcccctattttggatcagccactgatgaaaggtcggaagcccaactcgggtatgtaagaaatctaagcaaacccgcaaaggcggccatccggatagctatggtgtatacgtgggcctacgggactgatgagatatcgtggatggaagccgctcttatagcccaaacaagagctaatctgagcttagagaatctaaagctgctgactcctgtttcaacctccactaatctatctcataggttgaaagatacggcaacccagatgaagttctctagtgcaacactagtccgtgcaagtcggttcataacaatatcaaatgataacatggcactcaaagaagcaggggagtcgaaggatactaatctcgtgtatcagcagattatgctaactgggctaagcttgttcgagttcaatatgagatataagaaaggttccttagggaagccactgatattgcacttacatcttaataacgggtgctgtataatggagtccccacaggaggcgaatatccccccaaggtccacattagatttagagattacacaagagaacaataaattgatctatgatcctgatccactcaaggatgtggaccttgagctatttagcaaggtcagagatgttgtacacacagttgacatgacttattggtcagatgatgaagttatcagagcaaccagtatctgtactgcaatgacgatagctgatacaatgtctcaattagatagagacaacttaaaagagatgatcgcactagtaaatgacgatgatgtcaacagcttgattactgagtttatggtgattgatgttcctttattttgctcaacgttcgggggtattctagtcaatcagtttgcatactcactctacggcttaaacatcagaggaagggaagaaatatggggacatgtagtccggattcttaaagatacctcccacgcagttttaaaagtcttatctaatgctctatctcatcccaaaatcttcaaacgattctggaatgcaggtgtcgtggaacctgtgtatgggcctaacctctcaaatcaggataagatactcttggccctctctgtctgtgaatattctgtggatctattcatgcacgattggcaagggggtgtaccgcttgagatctttatctgtgacaatgacccagatgtggccgacatgaggaggtcctctttcttggcaagacatcttgcatacctatgcagcttggcagagatatctagggatgggccaagattagaatcaatgaactctctagagaggctcgagtcactaaagagttacctggaactcacatttcttgatgacccggtactgaggtacagtcagttgactggcctagtcatcaaagtattcccatctactttgacctatatccggaagtcatctataaaagtgttaaggacaagaggtataggagtccctgaagtcttagaagattgggatcccgaggcagataatgcactgttagatggtatcgcggcagaaatacaacagaatattcctttgggacatcagactagagcccctttttgggggttgagagtatccaagtcacaggtactgcgtctccgggggtacaaggagatcacaagaggtgagataggcagatcaggtgttggtctgacgttaccattcgatggaagatatctatctcaccagctgaggctctttggcatcaacagtactagctgcttgaaagcacttgaacttacctacctattgagccccttagttgacgaagataaagataggctatatttaggggaaggagctggggccatgctttcctgttatgacgctactcttggcccatgcatcaactattataactcaggggtatactcttgtgatgtcaatgggcagagagagttaaatatatatcctgctgaggtggcactagtgggaaagaaattaaacaatgttactagtctgggtcaaagagttaaagtgttattcaacgggaatcctggctcgacatggattgggaatgatgagtgtgaggctttgatttggaatgaattacagaatagctcgataggcctagtccactgtgacatggagggaggagatcataaggatgatcaagttgtactgcatgagcattacagtgtaatccggatcgcgtatctggtgggggatcgagacgttgtgcttataagcaagattgctcccaggctgggcacggattggaccaggcagctcagcctatatctgagatactgggacgaggttaacctaatagtgcttaaaacatctaaccctgcttccacagagatgtatctcctatcgaggcaccccaaatctgacattatagaggacagcaagacagtgttagctagtctcctccctttgtcaaaagaagatagcatcaagatagaaaagtggatcttaatagagaaggcaaaggctcacgaatgggttactcgggaattgagagaaggaagctcttcatcagggatgcttagaccttaccatcaagcactgcagacgtttggctttgaaccaaacttgtataaattgagcagagatttcttgtccaccatgaacatagctgatacacacaactgcatgatagctttcaacagggttttgaaggatacaatcttcgaatgggctagaataactgagtcagataaaaggcttaaactaactggtaagtatgacctgtatcctgtgagagattcaggcaagttgaagacaatttctagaagacttgtgctatcttggatatctttatctatgtccacaagattggtaactgggtcattccctgaccagaagtttgaagcaagacttcaattgggaatagtttcattatcatcccgtgaaatcaggaacctgagggttatcacaaaaactttattagacaggtttgaggatattatacatagtataacgtatagattcctcaccaaagaaataaagattttgatgaagattttaggggcagtcaagatgttcggggccaggcaaaatgaatacacgaccgtgattgatgatggatcactaggtgatatcgagccatatgacagctcgtaataattagtccctatcgtgcagaacgatcgaagctccgcggtacctggaagtcttggacttgtccatatgacaatagtaagaaaaacttacaagaagacaagaaaatttaaaaggatacatatctcttaaactcttgtctggt

SEQ ID NO: 5 - RNA, 14412 nt, Artificial
  a Sendai virus genomic RNA
accagacaagaguuuaagagauauguauccuuuuaaauuuucuugucuucuuguaaguuuuucuuacuauugucauauggacaaguccaagacuuccagguaccgcggagcuucgaucguucugcacgauagggacuaauuauuacgagcugucauauggcucgauaucaccuagugauccaucaucaaucacggucguguauucauuuugccuggccccgaacaucuugacugccccuaaaaucuucaucaaaaucuuuauuucuuuggugaggaaucuauacguuauacuauguauaauauccucaaaccugucuaauaaaguuuuugugauaacccucagguuccugauuucacgggaugauaaugaaacuauucccaauugaagucuugcuucaaacuucuggucagggaaugacccaguuaccaaucuuguggacauagauaaagauauccaagauagcacaagucuucuagaaauugucuucaacuugccugaaucucucacaggauacaggucauacuuaccaguuaguuuaagccuuuuaucugacucaguuauucuagcccauucgaagauuguauccuucaaaacccuguugaaagcuaucaugcaguuguguguaucagcuauguucaugguggacaagaaaucucugcucaauuuauacaaguuugguucaaagccaaacgucugcagugcuugaugguaaggucuaagcaucccugaugaagagcuuccuucucucaauucccgaguaacccauucgugagccuuugccuucucuauuaagauccacuuuucuaucuugaugcuaucuucuuuugacaaagggaggagacuagcuaacacugucuugcuguccucuauaaugucagauuuggggugccucgauaggagauacaucucuguggaagcaggguuagauguuuuaagcacuauuagguuaaccucgucccaguaucucagauauaggcugagcugccugguccaauccgugcccagccugggagcaaucuugcuuauaagcacaacgucucgaucccccaccagauacgcgauccggauuacacuguaaugcucaugcaguacaacuugaucauccuuaugaucuccucccuccaugucacaguggacuaggccuaucgagcuauucuguaauucauuccaaaucaaagccucacacucaucauucccaauccaugucgagccaggauucccguugaauaacacuuuaacucuuugacccagacuaguaacauuguuuaauuucuuucccacuagugccaccucagcaggauauauauuuaacucucucugcccauugacaucacaagaguauaccccugaguuauaauaguugaugcaugggccaagaguagcgucauaacaggaaagcauggccccagcuccuuccccuaaauauagccuaucuuuaucuucgucaacuaaggggcucaauagguagguaaguucaagugcuuucaagcagcuaguacuguugaugccaaagagccucagcuggugagauagauaucuuccaucgaaugguaacgucagaccaacaccugaucugccuaucucaccucuugugaucuccuuguacccccggagacgcaguaccugugacuuggauacucucaacccccaaaaaggggcucuagucugaugucccaaaggaauauucuguuguauuucugccgcgauaccaucuaacagugcauuaucugccucgggaucccaaucuucuaagacuucagggacuccuauaccucuuguccuuaacacuuuuauagaugacuuccggauauaggucaaaguagaugggaauacuuugaugacuaggccagucaacugacuguaccucaguaccgggucaucaagaaaugugaguuccagguaacucuuuagugacucgagccucucuagagaguucauugauucuaaucuuggcccaucccuagauaucucugccaagcugcauagguaugcaagaugucuugccaagaaagaggaccuccucaugucggccacaucugggucauugucacagauaaagaucucaagcgguacacccccuugccaaucgugcaugaauagauccacagaauauucacagacagagagggccaagaguaucuuauccugauuugagagguuaggcccauacacagguuccacgacaccugcauuccagaaucguuugaagauuuugggaugagauagagcauuagauaagacuuuuaaaacugcgugggagguaucuuuaagaauccggacuacauguccccauauuucuucccuuccucugauguuuaagccguagagugaguaugcaaacugauugacuagaauacccccgaacguugagcaaaauaaaggaacaucaaucaccauaaacucaguaaucaagcuguugacaucaucgucauuuacuagugcgaucaucucuuuuaaguugucucuaucuaauugagacauuguaucagcuaucgucauugcaguacagauacugguugcucugauaacuucaucaucugaccaauaagucaugucaacuguguguacaacaucucugaccuugcuaaauagcucaagguccacauccuugaguggaucaggaucauagaucaauuuauuguucucuuguguaaucucuaaaucuaauguggaccuuggggggauauucgccuccuguggggacuccauuauacagcacccguuauuaagauguaagugcaauaucaguggcuucccuaaggaaccuuucuuauaucucauauugaacucgaacaagcuuagcccaguuagcauaaucugcugauacacgagauuaguauccuucgacuccccugcuucuuugagugccauguuaucauuugauauuguuaugaaccgacuugcacggacuaguguugcacuagagaacuucaucuggguugccguaucuuucaaccuaugagauagauuaguggagguugaaacaggagucagcagcuuuagauucucuaagcucagauuagcucuuguuugggcuauaagagcggcuuccauccacgauaucucaucagucccguaggcccacguauacaccauagcuauccggauggccgccuuugcggguuugcuuagauuucuuacauacccgaguugggcuuccgaccuuucaucaguggcugauccaaaauaggggauucuuauagccggacauccguuuguaagcguguccaggucuauagagucaggaagauagaaccauguauagauggggucugcuccuucagaccugcaaagcuugcacaccucugaaccuucgauaaauauuccccucaagagcucuaaagggucugguguuucuagcccauguaugggucucccguaagucagguggauccacauuuucugccuuagaccgacagcuagcucaacugaacacauauacucauauucgauguugucuuucaccgguuuccugagaguucuagucagugucucguacugcaauagaucauaauugacaagccuccucaauaucccauaugauaauccuccuuuccuaacgcuggcucucacuagagacuuggucguaucaagcaucccugcaaucgccucccuaacuccaguuaaggaauuacccaggaucucaugagccacucucggcaggaugacuuuccgguccauaaggaacgaggccagguugagauccucuucuccacuagucucggugaagagaccagacaguagaggauucggggauuccugcagcacagaucuagcagugauauucuuuauaaucguaguuauacucugagaaugcgggagguuacacgaauaagggucugaagcccaaucuagaaaacuagagucaccggguucuugauucaugacccuguauaauaccugcuugucuaacagauccgcucugaugaaucuuuugagaucagcuagggcugcuacugcggggucuccaauauuucuaacaaagcaucuagauguagacauguaguugaauccuccaacauuugcuggaaucaacacugcacaucucagccaauucuuacccuuaaaguauugaucucuuacggucgggcugauaguuggauuuauagucaucccuagugauaugcacaccugcugacaggucuuauacaacgcaaugcaguagccuaguauaggagaauacccauuuucgauagcuuuugcuauggauguugagauguucgaacaagcagaucuguuuucaucuaccagugucucggaccagaauacacacuuggucaaggcuuucaggcacugugguaaaaucuucccaucauaguauauccuuuuacuauagacaaacaucuugcuacuaaugauggucucguucaauuuuagcucgugcccuacaucaaacaugacgugucuuagagcaccgaaauauuuggugaucuccucauagacaugauuuuucuucugcuuguaagucugagcuacagguacucuugaugucacggcuauagcuugauugucacccugaaccauugcagagacccugacacccacucucacagcugcuagguggauugcacugauugagauuaagguccacagcuucuggcaguaaccuucuaugcccccccuaggauuauguaugaaaaugccagagucugcaugauccuggaguugucgaugcauccggucggcgacuggacaguaaggaucuccaacauauauuguacaccuuucaaggacuggaugcauccaguuaaagaaggucuugaagccaaauaucucguugcaucucugaccaaacaaugcaguacucucaaaucuccaguuuaagcaguauuucuugaggucuguugugaggaagcaacuuaacguuucauagccgucuguugaugaaucuguugccuugaauucaugucuggaccucuucuuuucgucccaguaccccccagaguucuuauuuuccaugccuucguuucucuucucugaugauuuagaguuauuguacacugaaucaguccuggggacgccugagacagaaagaguagucaaucuuuuaaguaggucuaucucuccuuuaaccaucccauuuucgcugaauagcucuccuauuccuuuagccaguagugucucugccagcaccuguacggcucgcaucuuauaagucauuuuugcgaauagacgacccucuugcuugaucucuuucucuuugagacuguacgagauguugaacuccucgucuuucaaccaaucuccugacuccacauaauugauaauuucuucuggguugaaauucucaucauuuaugaacacuucaauaagccgccgggucucuucagacucuggggcuuuauaguacagauuacuauccggguauacagagucccaugccuccuuccugggggauagugcuuugucuuucauauauauugugagaucuucaucuaguugugguucuauaaacuuccgaaacuugaagccuaugaaacuuguauaguugucuacagcacauucauaagagauugccguauuggacccuugagcguuccuuaguucuagacacacgugaucagggaagucacaggggggccacuguccgccaugccucucucuauacccauuuaugaugauagugcaaaaaacugcaugacacucguauagggucuuaagcuuuauugccuuuugugcauacauaugggcccuuaccuugucggcggcagugacagccucuaagcugggguggccaaauguccuaaagaaggaaaagaucucugcuuucucaucaauagagguuccauggaaaauggcgaguaacgacuccacaauagugucugcuucagcaucuguguacacgucucuacuuguuaaaacagucuguagcucugucaacacaugccucauaaaugccccacguagagguauaacaggaucauuuaguuguaugagagcaagugauaggggcuccaauagugcgaugacauuguauauuuccucuccaagacuugagaagagggaauccacuaguucccauaauuccucaccuuugcuuguuaucccaauggacuucuuaucuagaugcccugcagcagacauauuccaccuuccuucuacaacaucacaauacaucaagaccagcucagggguuaggauauacccugucaaugucaacuuguucaguaucaugacaagaucuccguauguuacuagaguguaugauuugcauucuaggagguuaugugaauuagagguaucgagggguccccccggucuggucuucugcauccaccgcaugucauauuugaugcugaaccaaguuaggaauggccuauaccaucuauuccugcuguacuuaucaguuaucuccgggauggugccgauauccugcaacggaucguaccccucucuuccuucuauauugccuaguugcuugaagauauucaaccaaagauccuggaacccacuagauaacucccuggucagccgauccgagaccgcgaagacggaucggauuuugucacauaucucugguauaucaagccuaaguaauuccugagaguagguuggguacgguucaaacguguaucggucuaaauccuuuauugugcguugaagagccuuacccagagaccugaucuuaauuugacggggggacaauccuccguuccuaauuuugugcuuuguaauauuuauuaugcugucguccuucagucuguagggcugguucacaucuaacaagacgugcaacugugcuaucuucccccugacuaugggagaguucagguggcauucuggauagaguaugucagaaggguuuugggaggacuccugcccauccaugaccuauggcaagcuucccauucacccuggguuuuucuuaauacugugagagacguaagagacugagagauauuguggauguucggagaugacucuagugucagcaaggccgacaagccugcuagucaguuaaauuuaagacucggccuugcauaauuuagggaugcuagucuuaaagagcaucggcuguaagguauucaggcucuucugauugaucucgaugaugugaaagcaguagccuuuaccaaaaugcgugauacacgaugucgugguauaugcagccucuaauugaacauccuuuauccuuaacauauuuauaauguuaguaguguuagaauacaugauuguuggguugacacgcgauguauuggcauauagcgugacgguagcgacguuagcugcaucaggggacaauggauaagcaucaguguauacgccugauaugcauuccuucggacacuuauuguaccaauugcacucuucauuuccuggucuagacaaggcuucaugagguguccaguugauagucaaaggguggcugacaucaaguacuccuaucugcaguugagagugccagccugaugaucuuguauagauguacacccgaucacccaauuuuaauaaucuaccuuccgccccgagauaguuuugagugauuggaaugguugugacucuuaucuuuggccucucugagagauagucauugaccuggaugaucacgcugaccaccuguuucccuccuagccauguaauuuucagagccucauugcaugugucuugcgacaccuguuggcauccuuggguccuacauuuuguaucacccugcagaggggugguuaguccaccauacccaagaaauaucaaugagccuucuguugcaaugccguugccuacacugggguauagugcagagaacgggugaucaagaucuaccucgcuguugcgauaccggugagacuuaguucucccuuugagauccaggacaucaaggaccagauccucaauaccaucacuagaguagucgguucuuucgucuacagucggcauggagcaaagcugauaaccccuaguccggguugucaccacagagcaugauuuccgauugucguugaugucauaagugugggacacuacgggguuaagaucagggaacauaucugaauugagugauauguacccuagcugcaggaccugauaugauuucccuaugucagcacaaccuuguguaaugagauuugaugaauaggcauagauugccucgccaauugagagugaagggagccuaacacauccagagaucguuguagaaccagauaacaagcucggaccaggcagcaaugagauuucaggaucugagcuaagauacgguucuccgacagggcaucuccagaaacuauguggcucaaguggggcaauuccaucggcaugguggacugcgaucguacucucacagugcugagugagcucuugucugcugcacgacuuaucaaucaucuggaugacaucccugcuguuuuuguucaacaagacugggauuccgguuugcacagagcucugaauguugacagcccuugcuauaaccucuugccuuauuagacugguaagugacucuuucaccucccugcugcucauguucaaugccucuacagucauugaguacucuuucauacuauacccuugucuagcagaaauuaugauacagaugaucacuguggcaauugacaaagcccacugggugaaugagagaaucagcaaccaugugucggcuuuacuugaccucucccaaccugaugcugguuuuguggugcuaccacuaggagaaguagaccaguacgagucacguuugccccuaucaccauccaugaucugugcuuguuugaggcggccgcgaugaacuuucacccuaaguuuuucuuacuacgguuagcuuuucauuuugaucaucauguaugcuucuaugcaguugaugaagaugucaaauucauucauggccuuguagacaccuuggucuuggagcuuauuaaaaucacucuucaccugcuccacugccuugcucuuauuuucacaggggagaaaucgaugacagcgccucagccgcauccugagggucuucagcuucucacccagggaauucaaaugcuccuugauuucugggccaugcuucucugccuggggcaucacuucuaccagguaaaacuggaucauuuccgauaaggcuuggcaacccaaguaacccuuaaaguccugcauuaaggagucgguuagcaguauguuguccagcugguccuuuguuugaaagaaagucuucaccuggcugaaggcaguccgcagcucuaggagcauguggcucuggccgacugggaagugggugcaguuauugucuucccggcuguacuggccccugcugauccucaugccagucaguaagagcaggcagcauagcagugcugagccaggcauugaacuuuggcggccgcuuaauuaaccaagcacucacaagggacuuuaucccuaaguuuuucuuauuuaagacaaggagugacccgguggcuccgacugccagguggugucugcuuggggcauugucgcaggucucugaugggugcacauuuacagcuuucugauccuuccgauguucuuagccacaacauuaggguaguagcggaaaucacgagggauggccgguuggaacaccgcaucgacgccugugauuucuacagaugccgcccaaaucaccauguucauauggggauucacauccauuaaugggaagcagacugcccucuuccaugcgagcugacucaugaaugucuuagauagugucccauuaaccugaacauggaagcuuauaccgccgauuaacccaagugagaaaaucagccgcauucucucaaucuugcucuugcaguacucaacagaguauaucuucccgaccuuucuccugaucaacccgaggugcaccauaaaauugagcuuuuucuccccuugaucaucaaguacugggaguacccccuuuuguucuguggagaucccggucuugagugucaccaguaaauuaacagauauagaguugggcaaugcaaggucugcaagggucuuugggaucuuggauauggugauugccccuagagaugucccauugacaaacaccacucugagucuuauguccuuguccacagggaggcauuggggagcuagugcgaccuuguuugcauuaaauaucauucccugucucagccugccugaccaugguaggaguggagcaccaaucgaauccaccauguauacgaucaucucuccugcucgaacagcccuccucaccguaauucugagaucggugcaggccuuuaagaguuccugaucagucccguaguauuuggccacaccuauggguaacgacccggagccgcauauugaguagcuggucggcucugucaagucagauacgcucucuagauugguuguuuguuucggugucucaaagaaacccaagagcaauaaaucuagguaucucacuccauguuuaggagggucuccuaccuugacaauccugaugugggggauggcuuucuuauccggaccaguucucagaggcaggggcuccacaguaccguuauccucauaugagaacuuagggaaucuauagauaucugccauugcgccguguuaggugaaauuucuuucacccuaaguuuuucuuaaucuuuacuggcugaugcugaugguagauuggaucucucuaugacugaggaugguaggaugccucacccgggaucuaguuggucagugacucuauguccucuucuacgaguuccaugacugccuuaaccucuuggucugucuugcacuuggauaaugauuucacauaugcuacuuucucagcucugcuuaggggacugcucucuaugacgagccugagagagugcauugugggcuucucuuuggagggaaagagacgugaugcguuugaggcccuggguucugugucccucucuugguacaccggguugcggaucucaucucuaaauucauccucucggauuagguccgguuuguacuucauaucuucuagggucuccauagaugggucaaaccugguagccuuagucuuguucucuuuugauuuugcaaaaacggagggggaccuuguaagggagucuguguugucagucuugccaccucuaucugugaugauaugaaguguagauagguuggacaucagcaaugaguucuguucuuucugauacucagagaaucucuuguaaauaucccggaaugauuccacgcucucuuggaucuguuugagcaguuguuuguucucaucuaccuuacgagcggaagauuucucggcagaaaggaucaggccgcauacauugaaugucaucucugcauaguuugcagacuuuagggcacgucuugcaaacacauaacucgcgucucgggaugauucgaauucuugagcagacuggauuacaccaagacucgucaacaauguagccaucucuuucauagaugauguguucucuccuaugcccuuuuuuguugagucggucucuagacccggguggauugggcggccguuggcuggacaaucugagacagagcgggucccuauuggugguuuccggucuuugacccugacggcggggguguccccagaguugaugugcucauccuguguagauggggguuuuccugguggugacccugugcuguuguaacgauucagcgguggggaccgggugccaggcacgguugcuggaguaagagguuuggacccacuguugguaggucuucuuuuguuccuccguagcacagccucuucaaguucggggcuaggaaucaccaggaccccaguuacucuugcacuaugugagcugccaggcuccaugcuucuuccauuauuacuugcuccaccuucuccuucaucagguagggauguacuuccucguaccucuucuggaaguccuucagcuuggucuucuccccucuuaucagggugcgcagccaucucucuguuuucaucuucaauaccugaucucggauauccucucucauuuggaggauuuucaaggauuccggagucuccuccaucgcccagauccugagauacagaguuuguaccaguucuucccccaaaggcccgguguauauuuuguuuaucaagguuuccagcaugugcuucugccucuggcuugcuuguucucccagagacucuacucuccucaccugaucgauuaucuugggucgacgguguugagacuucuccuucgcccucacuuuuggcucuaugagcagagccugguccuuggggaguguugaugguguuguggagccagcuucuguccccuccgaugucaguugguucacucgacaggacagcaucgaggaauccgauaacauccgagagcgacucucguccuccuggcgccucccucucaacuucagaaucuucuuuaagaaugaaggcaucuugauccaugcgguaaguguagccgaagccguggcugucucgacugcuggguguggucggugggguuggguguggccuugccugagccgaucgguggaugaacuuucacccuaaguuuuucuuacuacggaucaaguaccuugaagccucguaugauccuagauuccuccuaucccagcuacugcugcggcaucgucaucuucgucaugaucgacaccguuauugcggccuucaucuccauggguugcagaauccucuugccgucucucugcgagucucauggcuauucuucucucuaugucugauacauccucaucauugguuuccuccucuaaccguucagccccauguagugugacaaaguggccaccacucaccugacgugcccaucuuucaccacuaucuccaccccaaccccuagcguccugguccgcaugggcuuuuguuucuaggucgaugucggcauugucuagagcuaccucaauugcaccaccgccuguugguuugugguaagcaccauccccaccggacaaguuugccagaugaugucugagccuccccuuggcuguauccgucacuccuaacucaucuuccaaggcacugcugaucuucgauucagcauccuuugccacggcuuguccuaguaagaacauuuccauaucaagguauguccucccugugacguacugcugcauugccuuguucuguacgacggcgacucccauggcguaacuccauagugcaggauaauugccuggagcaaauucaccaugaacaggguccuugaggauacagauaaagggagcucuggggccuuuugacagguaggugucuaugaggcuucuaagcuuauuaauaucgggccucagguuugacaacguuagagcugccaucuuuguuuccaccccauauuuaauaguguucaugaaggaagccagcccugcaucucggauguaguucccaacgaucuggauguucuucucuaauguggugagaucagaucuugcaguauucauagucacaagggucucaaccaugagagauacaaggcuuugcugagaucucauaaccgagccuauccccucaacugucuccccagugaaaacuaaggcaccuuucacggugccgucuugucugaacgccucuaaccuguugaagaacccuuuccuuaagccggcgcugcuugugauggccuucaccagcacaauccagacuuggacaauuauugcuccuaggcaugcaggauacccauagauuugaaggagugugucagggucugcagcaucccguugacccuggaagagugggcucuuguugaccauagguccaaacagccauucugugguccucucauauuccauaucucucgucuucacaaugaauccgucugucuucguccucuuagggucuuucucuauguuguagaucacauauuugacaucggcguuuacuccguuuguugucaaguacaauucuggacuacuguaagccauggcaagcagagagacgaggaaccccccucucugagagugcugcuuaucuguguccaaugagugagcuaggaagguaguugcaaugaauaacuugucugcaucaucagucacacuugggccuaguacgaacacugagacugugcuccucuggccggggauaacagcaccuccucccgacuuauuaauacuuucgcuccuccuagagcuaaauguaucgaaggugcucaacaacccggccaucgugaagaucugcggccgcgaugaacuuucacccuaaguuuuucuuacuacgguuaagucgcuaugacaacaccgcccaccaugaguccaaugauugcaccuuuguuugaacccacaucuucugcaaagaacaccaauuuuugaugaugaacuucauauccugagucaugucggaauucugcaucaagcgcccgagccguccaggcggccagcaggagcagugccaaaccgggcagcauaagugaaccuuggcggccgcgugaacuuuggcagcaaagcaaagggucuggaaccugcuccucaggguggauacuuugacccuaaaauccuguauaacuucauuacauaucccauacauguuuuuucucuuguuuggu

SEQ ID NO: 6 - RNA, 9 nt, Artificial
  a start (S) sequence of minus strand RNA virus
uuucacccu

SEQ ID NO: 7 - RNA, 9 nt, Artificial
  a start (S) sequence of minus strand RNA virus
uuugacccu

SEQ ID NO: 8 - RNA, 9 nt, Artificial
  a start (S) sequence of minus strand RNA virus
auucacccu

SEQ ID NO: 9 - DNA, 9 bp, Artificial
  a start (S) sequence of minus strand RNA virus
tttcaccct

SEQ ID NO: 10 - DNA, 9 bp, Artificial
  a start (S) sequence of minus strand RNA virus
tttgaccct

SEQ ID NO: 11 - DNA, 9 bp, Artificial
  a start (S) sequence of minus strand RNA virus
attcaccct

SEQ ID NO: 12 - DNA, 15 bp, Artificial
  an end (E) sequence of minus strand RNA virus
tttttcttactacgg

SEQ ID NO: 13 - DNA, 18 bp, Artificial
  a spacer sequence
cggccgcagatcttcacg

SEQ ID NO: 14 - DNA, 18 bp, Artificial
  a spacer sequence
atgcatgccggcagatga

SEQ ID NO: 15 - DNA, 18 bp, Artificial
  a primer for amplifing a Sendai virus genomic fragment
gttgagtactgcaagagc

SEQ ID NO: 16 - DNA, 42 bp, Artificial
  a primer for amplifing a Sendai virus genomic fragment
tttgccggcatgcatgtttcccaaggggagagttttgcaacc

SEQ ID NO: 17 - DNA, 18 bp, Artificial
  a primer for amplifing a Sendai virus genomic fragment
atgcatgccggcagatga

SEQ ID NO: 18 - DNA, 21 bp, Artificial
  a primer for amplifing a Sendai virus genomic fragment
tgggtgaatgagagaatcagc

SEQ ID NO: 19 - DNA, 50 bp, Artificial
  an artificially synthesized primer sequence
attgcggccgccaaggttcacttatgctgcccggtttggcactgctcctg

SEQ ID NO: 20 - DNA, 81 bp, Artificial
  an artificially synthesized primer sequence
attgcggccgcgatgaactttcaccctaagtttttcttactacggttaagtcgctatgacaacaccgcccaccatgagtcc

SEQ ID NO: 21 - DNA, 248 bp, Artificial
  an artificially synthesized sequence
gcggccgccaaggttcacttatgctgcccggtttggcactgctcctgctggccgcctggacggctcgggcgcttgatgcagaattccgacatgactcaggatatgaagttcatcatcaaaaattggtgttctttgcagaagatgtgggttcaaacaaaggtgcaatcattggactcatggtgggcggtgttgtcatagcgacttaaccgtagtaagaaaaacttagggtgaaagttcatcgcggccgc

SEQ ID NO: 22 - DNA, 51 bp, Artificial
  an artificially synthesized primer sequence
acttgcggccgccaaagttcaatgcctggctcagcactgctatgctgcctg

SEQ ID NO: 23 - DNA, 79 bp, Artificial
  an artificially synthesized primer sequence
atccgcggccgcgatgaactttcaccctaagtttttcttactacggttagcttttcattttgatcatcatgtatgcttc

SEQ ID NO: 24 - DNA, 596 bp, Artificial
  an artificially synthesized sequence
gcggccgccaaagttcaatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaccgtagtaagaaaaacttagggtgaaagttcatcgcggccgc

SEQ ID NO: 25 - DNA, 35 bp, Artificial
  an artificially synthesized primer sequence
aagcggccgcctcaaacaagcacagatcatggatg

SEQ ID NO: 26 - DNA, 34 bp, Artificial
  an artificially synthesized primer sequence
aggcggccgcttaattaaccaagcactcacaagg

SEQ ID NO: 27 - protein, 43 aa, Homo sapiens
DAEFRHDSGYEVHHQKLVFFAEDVGSNKGAIIGLMVGGVVIAT

SEQ ID NO: 28 - protein, 61 aa, Artificial
  a polypeptide containing an A-beta fragmnet
MLPGLALLLLAAWTARALDAEFRHDSGYEVHHQKLVFFAEDVGSNKGAIIGLMVGGVVIAT

SEQ ID NO: 29 - DNA, 35 bp, Artificial
  an artificially synthesized primer sequence
tgcttggttaattaagctttcacctcaaacaagca

SEQ ID NO: 30 - DNA, 35 bp, Artificial
  an artificially synthesized primer sequence
tgcttggttaattaagcggccgcctcaaacaagca

(19) United States
(12) Patent Application Publication
(10) Pub. No.: US 2009/0170798 A1
(43) Pub. Date: Jul. 2, 2009

(54) Highly safe intranasally administrable gene vaccines for treating alzheimer's disease

(75) Inventors: Hideo Hara, Aichi (JP); Takeshi Tabira, Aichi (JP); Yoshikazu Yonemitsu, Fukuoka (JP); Makoto Inoue, Ibaraki (JP); Yumiko Tokusumi, Ibaraki (JP); Mamoru Hasegawa, Ibaraki (JP)

Correspondence Address: CLARK & ELBING LLP, 101 FEDERAL STREET, BOSTON, MA, 02110, US

(73) Assignees: JAPAN AS REPRESENTED BY PRESIDENT OF NATIONAL CENTER FOR GERIATRICS AND GERONTOLOGY, Obu-shi (JP); DNAVEC CORPORATION, IBARAKI (JP)

(21) Appl. No.: 11/918,862

(22) PCT Filed: Apr. 20, 2006
(86) PCT No.: PCT/JP2006/308913

§ 371 Date: May 22, 2008

Related U.S. Application Data
(60) Provisional application No. 60/727,690, filed on Oct. 18, 2005.

(30) Foreign Application Priority Data
Apr. 20, 2005 (JP) 2005-123015

(51) Int. Cl. A61K 31/7105; C12N 15/74; A61P 25/28

(52) U.S. Cl. 514/44; 435/320.1

(57) ABSTRACT

An objective of the present invention is to provide a safe and effective vaccine therapy for Alzheimer's disease. A minus strand RNA viral vector carrying amyloid gene was constructed, and administered intranasally to 24- to 25-months-old APP transgenic mice. The level of serum anti-A 42 antibody was determined and showed to be markedly higher than the control. The results of histological investigation showed that the administration of a vector of the present invention markedly reduced senile plaques in all of the frontal lobe, parietal lobe, and hippocampus. The brain A level was also markedly reduced. Furthermore, the administration of a vector of the present invention did not result in lymphocyte infiltration in the central nervous system.

### TECHNICAL FIELD

The present invention relates to vaccines based on minus strand RNA virus vectors for treating Alzheimer's disease.

### BACKGROUND ART

In the rapidly aging society of Japan, senile dementia is becoming a serious social problem including the problem of nursing those affected. Indeed, it has been reported that approximately 10% of the people aged 65 years or older in Japan have senile dementia. While Alzheimer's disease, one of the two major causes of dementia, accounts for approximately 50% of the affected population, there is no effective therapeutic method available to date.

Pathological findings of Alzheimer's disease have three characteristics: neuronal atrophy/loss; senile plaque formation due to aggregation and deposition of amyloid β (hereinafter, sometimes also abbreviated as “Aβ”) proteins; and neurofibrillary tangles due to abnormal Tau protein. The major amyloid β proteins generated in the brain of Alzheimer's disease patients are of 40 to 43 amino acids resulting from cleavage of amyloid precursor proteins by β and γ secretases. Senile plaque is an aggregation composed of central amyloid β and surrounding microglia, fibrous astroglia, and dystrophic neurites. At present, the most convincing pathogenic hypothesis for Alzheimer's disease is “amyloid cascade hypothesis”, suggesting the cause of the disease to be senile plaque formation by amyloid β aggregation and deposition.

Vaccine (immune) therapy based on this hypothesis is attracting attention as a novel therapeutic method for Alzheimer's disease. Vaccine therapy for Alzheimer's disease is a method of eliminating amyloid β from the brain by immunological techniques. Schenk et al. of Elan Corporation intramuscularly administered pre-aggregated Aβ42 in combination with an adjuvant to PDAPP-transgenic mice and confirmed a decrease of amyloid deposition in the brain (Patent Document 1 and Non-patent Document 1). In the clinical trials conducted by Elan Corporation and Wyeth Corporate, a synthetic Aβ42 peptide (AN-1792) was injected intramuscularly in combination with an adjuvant (QS21), and anti-Aβ antibody recognizing β sheet structure of amyloid was detected in the serum of the subjects (Non-patent Document 2). Improvements of higher brain functions were also reported (Non-patent Document 3). Unfortunately the Phase II clinical trial was discontinued because 6% of the patients (18of 298 patients) developed meningoencephalitis as an adverse effect, and one death was reported. Post-mortem pathological examination of the brain tissue of the patient confirmed absence of senile plaques in the neocortex, suggesting the effectiveness of the vaccine. Figures showing microglial phagocytosis of Aβ degradation products were observed in the regions where senile plaques had disappeared. This finding suggests that the microglia phagocytoses antibody bound to Aβ via Fc receptor.

The anti-Aβ antibody described above has been found to be non-reactive to neurons, glial cells, amyloid precursor proteins (APPs), and intracellular Aβ. Therefore, it is unlikely that the adverse effect described above results from brain inflammation caused by the antibody. To administer AN-1792 vaccine, an adjuvant is required. Adjuvant has a strong activation effect on immunity and induces T lymphocyte-mediated cellular immunity. It is possible that the adverse effect described above is an experimental allergic encephalomyelitis-like meningoencephalitis, which is caused by the infiltration of Aβ- or APP-reactive Th1 type CD4⁺ T cells in the brain due to adjuvant-induced cellular immunity (Non-patent Document 4).

Thus the pathological effect of a vaccine therapy using an Aβ peptide has been confirmed with AN-1792 vaccine. However, for the clinical application of vaccines, reduction of adverse effects such as meningoencephalitis is essential. Based on this view, improved vaccination methods have been devised, including an immunization method that involves the combined use of an Aβ peptide N-terminal fragment linked to a carrier protein recognizable by T cells and a Th2 adjuvant (Patent Document 2), and an adenoviral vector that comprises a nucleic acid encoding a fused protein consisted of an Aβ peptide and cholera toxin B subunit (Patent Document 3). Furthermore, the present inventors developed a highly safe oral vaccine, an adeno-associated viral vector comprising a DNA that encodes a peptide fragment of an Aβ peptide to induce humoral immunity (Patent Document 4). The above-mentioned adeno-associated viral vector vaccine does not require any adjuvant because it utilizes the intestinal mucosal immune system where Th2 T cells can be easily induced. In addition, such excellent characteristics have been confirmed by the experiments using APP transgenic mice that a relatively long lasting antigen presentation as 6 months in the intestinal tract is available with a single administration; it has an effect of reducing brain amyloid deposition and senile plaque formation; there is absence of any observable inflammation in other organs, and so on. Nevertheless, vaccines with even better safety and effectiveness are needed for the practical application of vaccine therapy for Alzheimer's disease.
Patent Document 1: WO 99/27944
Patent Document 2: WO 02/096350
Patent Document 3: WO 2004/050876
Patent Document 4: WO 2004/111250
Patent Document 5: WO 00/70070
Patent Document 6: Japanese Patent Application Kokai Publication No. (JP-A) 2000-253876
Patent Document 7: WO 2001/072340
Non-Patent Document 1: Schenk D. et al., Nature 400: 173-177, 1999
Non-Patent Document 2: Hock C. et al., Nat. Med. 8: 1270-1275, 2002
Non-Patent Document 3: Hock C. et al., Neuron 38, 547-554, 2003
Non-Patent Document 4: Clinical Research (Rinsho to Kenkyu) 82(3): 439-444, 2005

### DISCLOSURE OF THE INVENTION

The present invention was achieved in view of the circumstances described above. An objective of the present invention is to provide highly safe and effective vaccine therapies for Alzheimer's disease.

In order to achieve the above objective, the present inventors conducted dedicated research aiming at development of novel vaccine therapies for Alzheimer's disease. The inventors are experienced in the development of vectors for gene introduction and applicable to gene therapy, using Sendai virus (SeV). Sendai virus is a minus (−) strand RNA virus. The virus is not integrated into chromosomes because it proliferates only in the host cell cytoplasm and does not have a DNA phase in their life cycle. The virus therefore ensures a high level of genetic safety when used as a vector for gene introduction. In developing Sendai virus-based vectors for gene introduction, the present inventors deleted gene(s) from the Sendai virus genome to further improve the safety and make the vectors suitable for target diseases. The inventors succeeded in improvement of the vectors to be safer and incapable of causing secondary infections by deleting the gene of a membrane fusion protein (F protein), which is involved in the viral invasion into host cells, from the genome and in establishing an efficient viral vector reconstruction system (WO 00/70070). In addition, the inventors successfully developed influenza vaccines (Japanese Patent Application Kokai Publication No. (JP-A) 2000-253876) and AIDS vaccines (WO 2001/072340) using the Sendai virus vector.

Th2 cytokines, such as IL-10, play important roles in Th1/Th2 balance closely associated with immune responses in the body. Th1 cells produce IFN-γ and such, and enhance cellular immunity. IFN-γ produced by Th1 cells suppresses IL-10 production in Th2 cells. Meanwhile, Th2 cells produce IL-10 and such, and enhance humoral immunity. IL-10 produced by Th2 cells suppresses the production of IFN-γ and such in Th1 cells. In an attempt to produce vaccines for Alzheimer's disease that would suppress cellular immunity-mediated adverse effects, such as encephalomeningitis, the inventors thought to introduce a gene encoding a Th2 cytokine together with Aβ into the above-mentioned Sendai virus vector. The inventors then constructed a Sendai virus vector encoding Aβ1-43 and IL-10 and tested its effects. First, the vector was administered intranasally to 24- to 25-month old APP transgenic mice. Anti-Aβ42 antibody levels in the serum were determined 4 weeks and 8 weeks after the treatment. Only low levels of anti-Aβ antibody were detected in the control group mice and the levels increased with time. In contrast, in the group of mice administered with the vector of the present invention, high levels of anti-Aβ antibody were detected in the blood, and the antibody levels were found to be slightly lower at 8 weeks than at 4 weeks. Histological examination showed that senile plaques in the frontal lobe, parietal lobe, and hippocampus were all markedly reduced upon the administration of the vector of the present invention. Further, the amount of Aβ in the brain tissue was determined with ELISA. The result showed a significant decrease of Aβ in the brain tissue. In addition, CD3 and Iba-1 were used as indices to examine whether the administration of this vector caused lymphocyte infiltration in the central nervous system. The result indicated that neither lymphocyte infiltration nor abnormal (inordinate) microglial activation occurred.

The above study revealed excellent characteristics of a minus strand RNA viral vector expressing an Aβ peptide. First, this vector exhibited a significant effect in reducing Aβ in Alzheimer's disease model mice with extremely old age. The Tg2576 mice used in the experiment described above were 24 to 25 months old and nearly at the end of their lives. In the studies conducted so far to assess treatment methods for Alzheimer's disease, mice with age of up to, at the most, 18 months were used. This is the first time that a therapeutic effect was confirmed in mice with such an old age. Such old-aged mice have an enormous amount of Aβ deposited in the brain. Thus, the vector of the present invention can be highly effective for patients with more advanced Alzheimer's disease. Second, the vector of the present invention was effective at a lower dosage and frequency of administration compared to conventional vaccine therapies. For example, when conventional methods using an Aβ peptide and an adjuvant in combination are adopted, multiple-dose administration is generally required to establish the immunity. Indeed, administration was carried out several times to around a dozen times in the clinical trials of AN-1792. In contrast, the vector of the present invention demonstrated significant effectiveness after a single-dose administration. Furthermore, in a vaccine therapy using an adeno-associated viral vector (WO 2004/111250), vaccination was carried out by the same single dose administration procedure used in the Examples described herein, and the dosage was 5×10¹¹ genomes/body. In contrast, the dosage applied in the Examples described herein was 5×10⁶ CIU (Cell Infectious Unit)/body (about 5×10⁷ genomes/body when converted to genome copy number). The dosages of these two vectors differ by as much as about 10⁴. Third, the vector administration in the Examples did not cause meningoencephalitis in the mouse model. Meningoencephalitis observed in the AN-1792 clinical trial was reportedly found in a mouse model as well (M. Shoji, et al., 44th Annual Meeting of Japanese Society of Neurology, May 15-17, 2003). Development of meningoencephalitis in normal non-model mice (C57B6 mice) immunized with an Aβ peptide and an adjuvant has also been reported (Furlan R, et al., Vaccination with amyloid-beta peptide induces autoimmune encephalomyelitis in C57/BL6 mice, Brain 126:285-291, 2003). The results described herein suggest that the vectors of the present invention are also safe when administered to humans.

As described above, the vector of the present invention exhibited a high Aβ-eliminating effect at a low dosage and frequency of administration. The effect of a Sendai virus vector-based AIDS vaccine was confirmed to be partly due to cellular immunity (WO 2001/072340). Although the vector used in the Examples was designed to purposely depend on humoral immunity rather than on cellular immunity to prevent adverse effects, the excellent vaccine effect demonstrated herein implies the importance of humoral immunity in vaccine therapies for Alzheimer's disease. Adverse effects can be reduced with making humoral immunity dominant through the use of a Th2 cytokine or Th2 adjuvant. That is, the present invention provides minus strand RNA viral vectors encoding Aβ and uses thereof, in particular, the inventions described below:

[1] A minus strand RNA viral vector comprising an amyloid β-encoding nucleic acid.
[2] The minus strand RNA viral vector of [1], further comprising a nucleic acid encoding a Th2 cytokine or a partial peptide thereof.
[3] The minus strand RNA viral vector of [2], wherein the Th2 cytokine or a partial peptide thereof is IL-10 or a partial peptide thereof.
[4] The vector of any one of [1] to [3], wherein the minus strand RNA viral vector is a Paramyxoviral vector.
[5] The vector of [4], wherein the Paramyxoviral vector is a Sendai virus vector.
[6] The vector of any one of [1] to [5] wherein the amyloid β is Aβ43 or a partial peptide thereof.
[7] The vector of any one of [1] to [6] wherein the amyloid β is derived from human.
[8] The vector of any one of [3] to [7] wherein IL-10 is derived from mouse or human.
[9] The vector of any one of [1] to [8] wherein the amyloid β is a polypeptide encoded by the nucleotide sequence of SEQ ID NO: 1, or a portion thereof.
[10] The vector of any one of [3] to [9] wherein IL-10 is a polypeptide encoded by the nucleotide sequence of SEQ ID NO: 2 or 3.
[11] The minus strand RNA viral vector of any one of [1] to [10] which comprises the nucleic acid of SEQ ID NO: 5.
[12] A composition which comprises the vector of any one of [1] to [11] and a pharmaceutically acceptable carrier.
[13] The composition of [12], further comprising (i) a nucleic acid encoding a Th2 cytokine or a partial peptide thereof in an expressible manner, (ii) a Th2 cytokine or a partial peptide thereof, or (iii) a Th2 adjuvant.
[14] The composition of [13], comprising a Th2 cytokine, a partial peptide thereof, or a Th2 adjuvant.
[15] The composition of [13], comprising a nucleic acid encoding a Th2 cytokine or a partial peptide thereof in an expressible manner.
[16] The composition of [15], wherein the Th2 cytokine is encoded by the minus strand RNA viral vector comprising an amyloid β-encoding nucleic acid.
[17] The composition of [15], wherein the Th2 cytokine is encoded by a vector other than the minus strand RNA viral vector comprising an amyloid β-encoding nucleic acid.
[18] The composition of any one of [12] to [17], wherein the composition is a pharmaceutical composition.
[19] A pharmaceutical composition for use in treating or preventing Alzheimer's disease, wherein the composition comprises the vector of any one of [1] to [18].
[20] The composition of [19], further comprising (i) a nucleic acid encoding a Th2 cytokine or a partial peptide thereof in an expressible manner, (ii) a Th2 cytokine or a partial peptide thereof, or (iii) a Th2 adjuvant.
[21] The pharmaceutical composition of any one of [18] or [20], for use in intranasal administration.
[22] A method for reducing deposition of amyloid β, wherein the method comprises a step of administering the vector of any one of [1] to [11] or a composition comprising the vector.
[23] A method for treating or preventing Alzheimer's disease, wherein the method comprises a step of administering the vector of any one of [1] to [11] or a composition comprising the vector.
[24] The method of [22] or [23], wherein the administration is intranasal administration.
[25] The method of [22] or [23], wherein the administration is intramuscular injection.
[26] The method of any one of [22] to [25], further a step of administering a Th2 cytokine, a partial peptide thereof, a Th2 adjuvant, or a vector encoding a Th2 cytokine or a partial peptide thereof.
[27] The method of [26], wherein a Th2 cytokine, a partial peptide thereof, or a Th2 adjuvant is administered.
[28] The composition of [26], wherein a vector encoding a Th2 cytokine or a partial peptide thereof is administered.

The present invention provides novel minus strand RNA viral vectors that can effectively reduce the level of amyloid β deposition at a low dosage and frequency of administration. The vector described in the Examples herein was confirmed not to induce inflammation in the central nervous system, and thus it is very unlikely that the administration of the vectors of the present invention will cause such encephalomeningitis as caused when conventional vaccines are administered. The vectors of the present invention can be used as extremely effective means for treating Alzheimer's disease.

The present invention relates to minus strand RNA viral vectors comprising a nucleic acid encoding amyloid β.

Herein, a gene refers to a genetic material or nucleic acid encoding a transcriptional unit. The gene may be an RNA or DNA. Herein, a nucleic acid encoding a protein is referred to as the protein's gene. The gene may not encode a protein and, for example, may encode a functional RNA, such as a ribozyme or an antisense RNA. The gene may be a naturally occurring or artificially designed sequence. Herein, “DNA” includes single-stranded and double-stranded DNAs. The phrase “encoding a protein” means that a polynucleotide comprises an ORF encoding the amino acid sequence of a protein either in a sense or antisense direction so as to express the protein under appropriate conditions.

Minus strand RNA viruses refer to viruses comprising minus strand RNAs (antisense strands complementary to the viral protein-encoding sense strands) as the genome. Minus strand RNA viruses are also called as negative strand RNA viruses. In particular, the minus strand RNA virus preferably used in the present invention is a single-stranded minus strand RNA virus (also referred to as a non-segmented minus strand RNA virus). The “single-stranded minus strand RNA virus” refers to a virus having single-stranded minus strand RNAs as the genome. Such viruses include viruses belonging to the families of, for example, Paramyxoviridae (including the genera such as Paramyxovirus, Morbillivirus, Rubulavirus, and Pneumovirus), Rhabdoviridae (including the genera such as Vesiculovirus, Lyssavirus, and Ephemerovirus), Filoviridae, Orthomyxoviridae (including Influenza virus A, B, and C, and Thogoto-like virus, etc.), Bunyaviridae (including the genera such as Bunyavirus, Hantavirus, Nairovirus, and Phlebovirus), and Arenaviridae.

Specific examples of minus strand RNA viruses that can be preferably used in the present invention include Sendai virus, Newcastle disease virus, Mumps virus, Measles virus, RS virus (Respiratory syncytial virus), rinderpest virus, distemper virus, Monkey parainfluenza virus (SV5), human parainfluenza type 1, 2, and 3 viruses, belonging to the Paramyxoviridae family; Influenza virus belonging to the Orthomyxoviridae family; Vesicular stomatitis virus and Rabies virus belonging to the Rhabdoviridae family; and such.

In the present invention, Paramyxoviruses are preferably used. A Paramyxovirus refers to a virus belonging to Paramyxoviridae or derivatives thereof. Paramyxoviruses are a group of viruses with non-segmented negative-strand RNAs as their genome, and include Paramyxovirinae (including Respirovirus also referred to as Paramyxovirus, Rubulavirus, and Morbillivirus), and Pneumovirinae (including Pneumovirus and Metapneumovirus). Specific examples of Paramyxoviruses applicable to the present invention are Sendai virus, Newcastle disease virus, Mumps virus, Measles virus, Respiratory syncytial virus (RS virus), Rinderpest virus, Distemper virus, Simian parainfluenza virus (SV5), and Human parainfluenza viruses 1, 2, and 3. More specifically, such examples include Sendai virus (SeV), Human parainfluenza virus-1 (HPIV-1), Human parainfluenza virus-3 (HPIV-3), Phocine distemper virus (PDV), Canine distemper virus (CDV), Dolphin molbillivirus (DMV), Pesle-des-petits-ruminants virus (PDPR), Measles virus (MV), Rinderpest virus (RPV), Hendra virus (Hendra), Nipah virus (Nipah), Human parainfluenza virus-2 (HPIV-2), Simian parainfluenza virus 5 (SV5), Human parainfluenza virus-4a (HPIV-4a), Human parainfluenza virus-4b (HPIV-4b), Mumps virus (Mumps), and Newcastle disease virus (NDV). A more preferred example is a virus selected from a group consisting of Sendai virus (SeV), Human parainfluenza virus-1 (HPIV-1), Human parainfluenza virus-3 (HPIV-3), Phocine distemper virus (PDV), Canine distemper virus (CDV), Dolphin molbillivirus (DMV), Peste-des-petits-ruminants virus (PDPR), Measles virus (MV), Rinderpest virus (RPV), Hendra virus (Hendra), and Nipah virus (Nipah). Viruses of this invention are preferably those belonging to Paramyxovirinae (including Respirovirus, Rubulavirus, and Morbillivirus) or derivatives thereof, and more preferably those belonging to the genus Respirovirus (also referred to as Paramyxovirus) or derivatives thereof. Examples of viruses of the genus Respirovirus applicable to this invention are Human parainfluenza virus-1 (HPIV-1), Human parainfluenza virus-3 (HPIV-3), Bovine parainfluenza virus-3 (BPIV-3), Sendai virus (also referred to as Murine parainfluenza virus-1), and Simian parainfluenza virus-10 (SPIV-10). It is most preferable that Sendai virus be used in the present invention. These viruses may be derived from natural strains, wild strains, mutant strains, laboratory-passaged strains, artificially constructed strains, or the like.

In the present invention, a “vector” is a carrier for introducing nucleic acids into cells. Minus strand RNA viral vectors are carriers derived from minus strand RNA viruses to introduce nucleic acids into cells. Minus strand RNA viruses such as SeV are excellent gene transfer vectors. Since minus strand RNA viruses do not have a DNA phase and carry out transcription and replication only in the cytoplasm of host cells, chromosomal integration does not occur. Therefore, they do not give rise to safety problems caused by chromosomal aberrations, such as canceration and immortalization. This characteristic of minus strand RNA viruses contributes greatly to safety when using a minus strand RNA virus as a vector. When used for a foreign gene expression, SeV hardly showed any nucleotide mutations even after multiple continuous passages, indicating high stability of its genome and long-term stable expression of inserted foreign genes (Yu, D. et al., Genes Cells 2: 457-466, 1997). The lack of a capsid structure protein in SeV yields further qualitative merits, such as flexibility in the size of genes to be inserted and in the packaging thereof. A transmissible SeV vector can introduce a foreign gene of at least 5.5 kb in size, and can simultaneously express two or more genes by adding transcription units.

Sendai virus is known to be pathogenic to rodents, causing pneumonia; however, it is not reported to be pathogenic in humans. This was supported by previous reports that intranasal administration of a wild type Sendai virus to non-human primates and humans did not cause severe adverse effects (Hurwitz, J. L. et al., Vaccine 15: 533-540, 1997; Slobod, K. S. et al., Vaccine 22: 3182-3186, 2004). Two points, namely “high infectivity” and “high expression level”, can be given as examples of the advantage. SeV vectors infect cells by binding to sialic acid in the glucolipids and glycoproteins of the cell membrane. This sialic acid is expressed in almost all mammalian and avian cells, giving rise to a broad spectrum of infection, i.e., high infectivity. When a transmissible SeV replicon-based vector releases viruses, they re-infect neighboring cells. Multiple RNPs are replicated in the cytoplasm of the infected cells and distributed into the daughter cells via cell division, and thus continuous expression can be expected. Further, SeV vectors can be applied to an extremely wide range of tissues. Furthermore, their characteristic expression machinery, wherein transcription and replication occur only in the cytoplasm, has been shown to express inserted genes at very high levels (Moriya, C. et al., FEBS Lett. 425(1): 105-111, 1998; WO 00/70070). Furthermore, SeV vectors made non-transmissible by deleting an envelope gene have been successfully recovered (WO 00/70070; Li, H O. et al., J. Virol. 74(14): 6564-6569, 2000). Thus, SeV vectors have been improved to further enhance their “safety” while maintaining their “high infectivity” and “high expression levels”.

A minus strand RNA viral vector of the present invention comprises the genomic RNA of a minus strand RNA virus. Genomic RNA refers to an RNA that has a function to form an RNP comprising certain viral proteins of a minus strand RNA virus, which cause the expression of genes in the genome. Thus the nucleic acid is replicated to form daughter RNPs. RNAs of a minus strand RNA virus encode genes as antisense sequences. In general, in a minus strand RNA viral genome, viral genes are arranged as antisense sequences between 3′-leader region and 5′-trailer region. Between ORFs of respective genes are a transcription ending sequence (E sequence)-intervening sequence (I sequence)-transcription starting sequence (S sequence), such that the RNA encoding the ORF of each gene is transcribed as an individual cistron. Genomic RNAs in a vector of the present invention comprise antisense RNA sequences encoding N (nucleocapsid, also referred to as nucleoprotein (NP))-, P (phospho)-, and L (large)-proteins, which are viral proteins essential for the expression of a group of genes encoded by the RNA, and for the autonomous replication of the RNA itself. The RNAs may also encode M (matrix) protein essential for virion formation. Further, the RNAs may encode envelope proteins essential for virion infection. Envelope proteins of a minus strand RNA virus include F (fusion) protein causing cell membrane fusion, and HN (hemagglutinin-neuraminidase) or H (hemagglutinin) protein essential for viral adhesion to cells. However, HN protein is not required in infection to certain types of cells (Markwell, M. A. et al., Proc. Natl. Acad. Sci. USA 82(4): 978-982, 1985), and infection is achieved with F protein alone. The RNAs may encode envelope proteins other than F and/or HN proteins.

A minus strand RNA viral vector of the present invention may be, for example, complexes of the genomic RNAs of a minus strand RNA virus and viral proteins, that is, ribonucleoproteins (RNPs). RNPs can be introduced into cells, for example, in combination with a desired transfection reagent. Specifically, such RNPs are complexes comprising the genomic RNAs of a minus strand RNA virus, N protein, P protein, and L protein. On introducing RNPs into cells, cistrons encoding the viral proteins are transcribed from the genomic RNAs by the action of viral proteins, and at the same time, the genome itself is replicated to form daughter RNPs. Replication of a genomic RNA can be confirmed by RT-PCR, Northern blot hybridization, or the like to detect an increase in the RNA copy number.

Further, minus strand RNA viral vectors of the present invention are preferably minus strand RNA virus virions. “Virion” refers to a micro-particle comprising nucleic acids and released from cells by the action of viral proteins. Virions of a minus strand RNA virus have a structure in which the above-described RNP, comprising genomic RNAs and viral proteins, is enclosed in a cell membrane-derived lipid membrane (referred to as an envelope). Virions may have infectivity. Infectivity refers to the ability of virions to introduce carried nucleic acids into cells that they have adhered to, based on the cell adhesion and membrane-fusion abilities of the minus strand RNA viral vector. Minus strand RNA viral vectors of the present invention may be transmissible or non-transmissible vectors. “Transmissible” means that when a viral vector infects a host cell, the virus can replicate itself within the cell to produce infectious virions.

For example, each gene in viruses belonging to Paramyxovirinae is generally described as follows. In general, N gene is also described as “NP”.

| | | | | | | | |
|---|---|---|---|---|---|---|---|
| Respirovirus | NP | P/C/V | M | F | HN | — | L |
| Rubulavirus | NP | P/V | M | F | HN | (SH) | L |
| Morbillivirus | NP | P/C/V | M | F | H | — | L |

For example, the database accession numbers for the nucleotide sequences of each of Sendai virus genes are: M29343, M30202, M30203, M30204, M51331, M55565, M69046, and X17218 for N gene; M30202, M30203, M30204, M55565, M69046, X00583, X17007, and X17008 for P gene; D11446, K02742, M30202, M30203, M30204, M69046, U31956, X00584, and X53056 for M gene; D00152, D11446, D17334, D17335, M30202, M30203, M30204, M69046, X00152, and X02131 for F gene; D26475, M12397, M30202, M30203, M30204, M69046, X00586, X02808, and X56131 for HN gene; and D00053, M30202, M30203, M30204, M69040, X00587, and X58886 for L gene. Examples of viral genes encoded by other viruses are: CDV, AF014953; DMV, X75961; HPIV-1, D01070; HPIV-2, M55320; HPIV-3, D10025; Mapuera, X85128; Mumps, D86172; MV, K01711; NDV, AF064091; PDPR, X74443; PDV, X75717; RPV, X68311; SeV, X00087; SV5, M81442; and Tupaia, AF079780 for N gene; CDV, X51869; DMV, Z47758; HPIV-1, M74081; HPIV-3, X04721; HPIV-4a, M55975; HPIV-4b, M55976; Mumps, D86173; MV, M89920; NDV, M20302; PDV, X75960; RPV, X68311; SeV, M30202; SV5, AF052755; and Tupaia, AF079780 for P gene; CDV, AF014953; DMV, Z47758; HPIV-1, M74081; HPIV-3, D00047; MV, AB016162; RPV, X68311; SeV, AB005796; and Tupaia, AF079780 for C gene; CDV, M12669; DMV, Z30087; HPIV-1, S38067; HPIV-2, M62734; HPIV-3, D00130; HPIV-4a, D10241; HPIV-4b, D10242; Mumps, D86171; MV, AB012948; NDV, AF089819; PDPR, Z47977; PDV, X75717; RPV, M34018; SeV, U31956; and SV5, M32248 for M gene; CDV, M21849; DMV, AJ224704; HPN-1, M22347; HPIV-2, M60182; HPIV-3, X05303; HPIV-4a, D49821; HPIV-4b, D49822; Mumps, D86169; MV, AB003178; NDV, AF048763; PDPR, Z37017; PDV, AJ224706; RPV, M21514; SeV, D17334; and SV5, AB021962 for F gene; and, CDV, AF112189; DMV, AJ224705; HPIV-1, U709498; HPIV-2, D000865; HPIV-3, AB012132; HPIV-4A, M34033; HPIV-4B, AB006954; Mumps, X99040; MV, K01711; NDV, AF204872; PDPR, Z81358; PDV, Z36979; RPV, AF132934; SeV, U06433; and SV-5, S76876 for HN (H or G) gene. However, a number of strains are known for each virus, and genes comprising sequences other than those cited above exist due to differences in strains.

The ORFs of these viral proteins are arranged as antisense sequences in the genomic RNAs using the above-described E-I-S sequence. The ORF closest to 3′-end of the genomic RNAs only requires S sequence between 3′-leader region and the ORF, and does not require E and I sequence. Further, the ORF closest to 5′-end of the genomic RNA only requires E sequence between 5′-trailer region and the ORF, and does not require I and S sequences. Furthermore, two ORFs can be transcribed as a single cistron, for example, using an IRES sequence. In such a case, the E-I-S sequence is not required between these two ORFs. For example, in wild type Paramyxoviruses, a typical RNA genome comprises a 3′-leader region, six ORFs encoding N, P, M, F, HN, and L proteins, and a 5′-trailer region in the antisense order. As in wild type viruses, genomic RNAs of the present invention preferably have ORFs encoding N, P, M, F, HN, and L proteins arranged in this order with a preceding 3′-leader region and a succeeding 5′-trailer region; however, the gene arrangement is not limited to this. Certain types of minus strand RNA viruses do not comprise all the six viral genes, but even in such cases, it is preferable to arrange their viral genes as in the wild type described above. In general, vectors carrying N, P, and L genes can autonomously express genes from the RNA genome in cells, resulting in replication of the genomic RNAs. Furthermore, by the action of genes such as F and HN (or H) genes and M gene encoding envelope-constituting proteins, infectious virions are formed and released to the outside of cells. Thus, such vectors become transmissible viral vectors. A gene encoding a polypeptide that comprises Aβ or IL-10 may be inserted into a non-protein coding region in this genome as described below.

Further, a minus strand RNA viral vector of the present invention may lack any gene(s) of the wild type minus strand RNA virus. As long as the viral genome RNAs encode the necessary viral proteins (N, L, and P) for RNP reconstruction, it can replicate within cells and express genes even if it does not encode envelope-constituting proteins. For example, depending on the virus type, vectors deficient in at least one of the genes encoding envelope-constituting proteins such as F, H, HN, G, M, and M1 can be illustrated (WO 00/70055 and WO 00/70070; Li, H.-O. et al., J. Virol. 74(14): 6564-6569, 2000). For example, a vector that does not comprise M, F, or HN gene, or any combinations thereof, can be suitably used as a viral vector of the present invention. Such viral vectors can be reconstructed, for example, by supplying the product(s) of the defective gene(s) from the outside. The viral vectors thus prepared adhere to host cells to cause cell fusion as well as wild type viruses, but they cannot form daughter virions that have the same infectivity as the original virus because the vector genome introduced into the cells lacks any of the original viral genes. Therefore, such vectors are safe and useful viral vectors to introduce genes for only once. Examples of genes, that the genome may be defective of, are the F gene and/or HN gene. For example, viral vectors can be reconstructed by transfecting host cells with a plasmid vector expressing a recombinant minus strand RNA viral genome defective of F gene, along with an F protein expression vector as well as expression vectors for NP, P, and L proteins (WO 00/70055 and WO 00/70070; Li, H.-O. et al., J. Virol. 74(14): 6564-6569, 2000). Viruses can also be produced, for example, using host cells that have F gene incorporated into their chromosomes. When supplying these proteins from the outside, their amino acid sequences do not need to be the same as the original viral sequences, and a mutant or homologous gene from another virus may be used as a substitute, as long as the nucleic acid-introducing activity is the same as or greater than that of the natural type.

Further, a vector comprising an envelope protein other than that of the virus from which the vector genome was derived may be produced as a viral vector of the present invention. For example, when reconstructing a virus, a viral vector comprising a desired envelope protein can be generated by expressing an envelope protein other than the envelope protein of the original virus of the vector on a packaging cell. Such proteins are not particularly limited, and include envelope proteins of other viruses, for example, G protein of Vesicular stomatitis virus (VSV-G) (J. Virology 39: 519-528, 1981). Viral vectors of the present invention include a pseudo-type viral vector comprising an envelope protein such as VSV-G protein derived from viruses other than the virus from which the genome was derived. By designing such a viral vector that the envelope protein(s) is not encoded in the virus RNA genome, the protein(s) will not be expressed after the virion infected cells.

Furthermore, a viral vector of the present invention may be, for example, a vector with, on the envelope surface, a protein that can attach to a specific cell, such as an adhesion factor, ligand, receptor, antibody or fragment thereof; or a vector comprising a chimeric protein with such a protein in the extracellular domain and a polypeptide derived from a virus envelope in the intracellular domain. Thus, vectors targeting specific tissues can also be produced. Such proteins may be encoded in the viral genome, or supplied by expressing the genes other than the viral genome (for example, other expression vectors or genes existing in the host cell chromosome) at the time of viral vector reconstruction.

Further, in vectors of the present invention, any viral gene comprised in the vectors may be modified from the wild type gene in order to reduce the immunogenicity caused by the viral protein, or to enhance RNA transcriptional or replication efficiency, for example. Specifically, for example, in a minus strand RNA viral vector, modification of at least one of the replication factors, N, P, and L genes, is considered to enhance transcription or replication efficiency. Further, HN protein, an envelope protein, has both hemagglutinin activity and neuraminidase activity. So, it is possible, for example, to improve viral stability in the blood if the former activity can be attenuated, and to control infectivity if the latter activity is modified. Further, it is also possible to control membrane fusion ability by modifying F protein. For example, analysis of antigen-presenting epitopes of F protein and HN protein, both potential cell surface antigenic molecules, enables to make viral vectors with reduced antigenicity of these proteins. Furthermore, a temperature-sensitive mutation may be introduced into a viral gene to suppress the release of secondarily released particles or virus like particles (VLPs) (WO 2003/025570). For example, mutations such as G69E, T116A and A183S, can be introduced into M gene; A262T, G264, and K461G into HN gene; L511F into P gene; and N1197S and K1795E into L gene. Temperature-sensitive mutations that can be introduced are not limited to the mutations described above (see WO 2003/025570).

Furthermore, vectors of the present invention may be defective of accessory genes. For example, by knockout of V gene, one of the SeV accessory genes, the pathogenicity of SeV toward hosts such as mice is remarkably reduced without hindering gene expression and replication in cultured cells (Kato, A. et al., J. Virol. 71: 7266-7272, 1997; Kato, A. et al., EMBO J. 16: 578-587, 1997; Curran, J. et al., WO 01/04272, EP1067179). Such attenuated vectors are particularly useful as nontoxic viral vectors for in vivo or ex vivo gene transfer.

Vectors of the present invention comprise an Aβ-encoding nucleic acid as a foreign gene within the genome of a minus strand RNA viral vector described above. There is no limitation on the number of amino acids encoding Aβ. For example, Aβ1-40, Aβ1-42, and Aβ1-43 are all acceptable. Any desired fragment having the antigenicity of a naturally occurring full-length Aβ or any polypeptide comprising the fragment can also be used as Aβ. To exhibit antigenicity against Aβ, such polypeptides must comprise six or more consecutive amino acids, preferably seven or more consecutive amino acids, or eight or more consecutive amino acids, derived from the amino acid sequence of naturally occurring Aβs, for example, Aβ43 (Harlow, Antibodies: A laboratory Manual, 1998; Chapter 5 page 76). For example, the majority of B cell epitopes against Aβ42 (1 to 42 in SEQ ID NO: 27) are concentrated in the 1st to 15th amino acids of N-terminal region (Cribbs, D. H. et al., Int. Immunol. 15(4): 505-14, 2003). Thus, a polypeptide comprising the 1st to 15th amino acids of Aβ42 can be suitably used as an Aβ peptide to be expressed from the vector. Alternatively, a polypeptide comprising the 1st to 21st amino acids or the 4th to 10th amino acids of Aβ42 may be expressed (JP-A No. 2005-21149). In addition, the anti-Aβ monoclonal antibodies 10D5 and 6C6, known for their ability to inhibit amyloid fibril formation and to protect neurons, have been reported to recognize a four amino acid-epitope corresponding to the 3rd to 6th amino acids of Aβ42 (EFRH; 3 to 6 in SEQ ID NO: 27) (Frenkel, D. et al., J. Neuroimmunol. 88: 85-90, 1998; Frenkel, D. et al., J. Neuroimmunol. 95: 136-142, 1999). The monoclonal antibody 508F that recognizes the same epitope also suppresses Aβ neurotoxicity (Frenkel, D. et al., J. Neuroimmunol. 106: 23-31, 2000). Thus, a polypeptide of 6 to 8 amino acids or more in the Aβ sequence including this sequence can be suitably used. Epitopes corresponding to cellular immunity are concentrated in the C-terminal region of Aβ. Accordingly, humoral immunity can be made relatively dominant over cellular immunity by expressing a fragment, which comprises an N-terminal fragment such as Aβ1-21, but not sequences near to the C-terminal as Aβ22-43 (the 22nd to 43rd amino acids of SEQ ID NO: 27). A vector encoding a polypeptide that comprises the sequence of naturally occurring Aβ peptides (Aβ39, Aβ40, Aβ41, Aβ42, and Aβ43 corresponding to respectively 1-39, 1-40, 1-41, 1-42, and 1-43 amino acids of SEQ ID NO: 27) can be suitably used as a vector of the present invention. Short Aβ peptide fragments may be used for making polypeptides fused with an appropriate carrier protein to increase their immunogenicity. Carrier proteins include keyhole limpet hemocyanin (KLH), bovine serum albumin (BSA), rabbit serum albumin (RSA), human serum albumin (HSA), ovalbumin (OVA), thyroglobulin (TG), and such. The short peptides can be fused with a carrier protein via a spacer of about 1 to 5 amino acids. The length (amino acid length excluding the carrier protein portion) of an antigen polypeptide (a mature polypeptide when it is in a secretory form) preferably ranges from 10 to 200 amino acids, more preferably from 15 to 100 amino acids, still more preferably from 20 to 80 amino acids.

Furthermore, an Aβ polypeptide encoded in a vector preferably comprises a secretion signal to ensure its secretion from the cells. As a secretion signal sequence, the signal sequence of a desired secretory protein, such as interleukin (IL)-2 or tissue plasminogen activator (tPA), can be used, and the N-terminal signal sequence of amyloid precursor proteins (APPs) is preferably used. Specifically, such secretion signal sequences include, for example, the secretion signal sequence of Accession number NP_{—}958817 (for the nucleotide sequence, see, for example, Accession number NM_{—}201414). In other words, a suitable vector of the present invention encodes an Aβ peptide to which a secretion signal has been attached.

In addition, vectors of the present invention may comprise nucleic acids encoding Th2 cytokines and/or anti-inflammatory cytokines as foreign genes. Th2 cytokines refer to cytokines produced predominantly by type 2 helper T cells (Th2 cells) rather than type 1 helper T cells (Th1 cells). Specifically, such Th2 cytokines include IL-4, IL-5, IL-6, IL-9, IL-10, and IL-13. In the present invention, a cytokine encoded in a vector is preferably selected from a group consisting of IL-4, IL-10, and IL-13, and most preferably IL-10 is encoded. The nucleotide sequence of each cytokine gene and its amino acid sequence are already known (IL-4: NM_{—}000589, NP_{—}000580, AAH66277, AAH67515, NP_{—}758858, NP_{—}067258, NP_{—}958427; IL-5: NM_{—}000879, NP_{—}000870, CAA31210, NP_{—}034688, NP_{—}068606; IL-6: NM_{—}000600, NP_{—}000591, XP_{—}518992, AAB30962, NP_{—}112445; IL-9: NM_{—}000590, NP_{—}000581, AAH66284, AAH66287, NP_{—}032399, XP_{—}341488; IL-10: NM_{—}000572, NP_{—}000563, CAG46825, NP_{—}034678, NP_{—}036986; IL-13: NM_{—}002188, NP_{—}002179, AAB01681, NP_{—}032381, NP_{—}446280).

A Th2 cytokine, such as IL-10, encoded in a vector may be a complete (i.e. wild-type) or partial peptide (i.e. active fragment) as long as it retains the activity. For example, the N-terminal signal sequence can be appropriately replaced by another signal sequence. Alternatively, the cytokine may be expressed as a fused protein with other peptides.

There is no limitation of the origin of Aβ and cytokines such as IL-10 encoded in a vector, which can be of any mammals including mice, rats, guinea pigs, rabbits, pigs, cattle, horses, donkeys, goats, dogs, chimpanzees, monkeys, and humans. The origins of Aβ and cytokines may be the same or different. It is appropriate to use Aβ and cytokines derived from a species that is the same as the subject of administration. Nucleotide sequences of the nucleic acids encoding such cytokines are available from the databases described above. Additional examples such as Genbank Accession NOs. AY410237 and NM_{—}010548 are available for mouse IL-10; and Genbank Accession NOs. AY029171 and NM_{—}000572 are available for human IL-10. An example of “Aβ-encoding nucleic acid” that can be suitably used as a vector of the present invention is human Aβ cDNA (SEQ ID NO: 1). Examples of “IL-10-encoding nucleic acid” that can be suitably used are mouse IL-10 cDNA sequence (SEQ ID NO: 2) and human IL-10 cDNA sequence (SEQ ID NO: 3). Nucleic acids having sequences different from the above nucleotide sequences of SEQ ID NOs 1 to 3 can also be suitably used for a vector of the present invention like the sequences of SEQ ID NOs 1 to 3, provided that they have the same amino acid sequences as the above-mentioned nucleotide sequences as a result of codon degeneracy. Insert region of the above foreign genes can be selected, for example, in a desired portion in the non-protein coded region in the genome. For example, they can be inserted between 3′-leader region and a viral protein ORF closest to 3′-end; between each of the viral protein ORFs; and/or between a viral protein ORF closest to 5′-end and 5′-trailer region. Further, in genomes defective of F or HN gene or the like, foreign gene(s) can be inserted into those defected regions. When introducing a foreign gene into a Paramyxovirus, the chain length of the polynucleotide to be inserted into the genome is preferably a multiple of six (J. Virology 67 (8): 4822-4830, 1993). A set of E-I-S sequences should be arranged between the inserted foreign gene and the viral ORF. Two or more foreign genes can be inserted in a tandem manner using E-I-S sequences. Alternatively, a desired foreign gene may be inserted using IRES.

Expression levels of a foreign gene, carried by a vector can be controlled depending on the type of a transcriptional initiation sequence added in the upstream (to the 3′-side of a minus strand) of the gene (WO 01/18223). Expression levels can also be controlled depending on the position at which the foreign gene is inserted in the genome: the closer the inserting position is to 3′-end of a minus strand, the higher the expression level; the closer the inserting position is to 5′-end, the lower the expression level. Thus, the position inserting a foreign gene can be appropriately adjusted to obtain a desired gene expression level, or to achieve the most suitable combination with viral protein-encoding genes in the vicinity of the foreign gene. In general, as it is thought to be advantageous to get high expression levels of Aβ, it is preferable to link a foreign gene encoding Aβ to a high-efficiency transcriptional initiation sequence and thus to insert it near to 3′-end of the minus strand genome. Specifically, a foreign gene is inserted between 3′-leader region and a viral protein ORF closest to 3′-end. Alternatively, a foreign gene may be inserted between an ORF of the viral gene closest to 3′-end and the next viral gene. In wild type Paramyxoviruses, the viral protein gene closest to 3′-end of the genome is N gene, and the second closest gene is P gene. Alternatively, when a high level expression of the introduced gene is undesirable, the level of gene expression from the viral vector can be suppressed in order to get an appropriate effect, for example, by inserting the foreign gene at a site in the vector as close as possible to 5′-side of the minus strand genome, or by selecting a low-efficiency transcriptional initiation sequence.

To express two polypeptides, one comprising Aβ and the other comprising a Th2 cytokine from a vector, the nucleic acids encoding the respective polypeptides are inserted into a vector genome. The two nucleic acids may be inserted into separate sites, or into a single site in a tandem manner via an E-I-S sequence. It is preferable to use a high-efficiency transcription initiation sequence as an S sequence. For example, 3′-UCCCACUUU-5′(minus strand RNA; SEQ ID NO: 6), 3′-UCCCAGUUU-5′(minus strand RNA; SEQ ID NO: 7), or 3′-UCCCACUUA-5′(minus strand RNA; SEQ ID NO: 8) can be suitably-used as the S sequence.

A vector of the present invention may carry another foreign gene at a position other than where genes encoding an Aβ and a Th2 cytokine have been inserted. Such foreign genes are not limited. For example, they may be marker genes for monitoring vector infection, or immune system-regulating genes such as cytokines and hormones. A vector of the present invention may carry one or more genes encoding such as signal transduction regulators, cytokines, hormones, receptors, antibodies, and fragments thereof. A vector of the present invention can introduce a gene either by a direct (in vivo) administration to a target site of a living body, or by an indirect (ex vivo) administration, in which a vector is infected to patient's cells or other cells and then the cells are injected into the target site.

Vectors of the present invention can be used as highly advantageous means for treating, preventing, or inhibiting the progression of Alzheimer's disease. A vector of the present invention exhibited a remarkable Aβ-eliminating effect in 24- to 25-month-old APP Tg mice, which generally have a large amount of accumulated Aβ. Since this effect was confirmed with only a single intranasal administration, vectors of the present invention can be a much less-invasive therapeutic method for patients. The finding that no inflammation was observed in the central nervous system also proves the vector highly safe to use.

To make a vector of the present invention, cDNA encoding the genomic RNA of a minus strand RNA virus of this invention is transcribed in mammalian cells in the presence of the viral proteins (i.e., N, P, and L proteins) essential for reconstruction of RNP that comprises the genomic RNA of a minus strand RNA virus (or a complementary strand thereof). The viral RNP can be reconstructed by generating either a minus strand genome (that is, same as the viral genome antisense strand) or a plus strand (the viral protein-encoding sense strand) via transcription. Generating a plus strand is preferable to increase the efficiency of the vector reconstruction. The RNA termini preferably reflect the termini of 3′-leader sequence and 5′-trailer sequence as accurately as possible, as in the natural viral genome. To accurately regulate 5′-end of the transcription products, for example, a sequence recognizable by T7 RNA polymerase may be used as a transcription initiation site and the RNA polymerase may be expressed within a cell. To regulate 3′-end of the transcription products, for example, an autocleavage-type ribozyme may be encoded at 3′-end of the transcription products to allow accurate cleavage of 3′-end by this ribozyme (Hasan, M. K. et al., J. Gen. Virol. 78: 2813-2820, 1997; Kato, A. et al., EMBO J. 16: 578-587, 1997; and Yu, D. et al., Genes Cells 2: 457-466, 1997).

For example, based on the descriptions by Hasan, M. K. et al., J. Gen. Virol. 78: 2813-2820, 1997; Kato, A. et al., EMBO J. 16: 578-587, 1997; Yu, D. et al., Genes Cells 2: 457-466, 1997; or the like, a recombinant Sendai virus vector carrying a foreign gene can be constructed as follows;

First, a DNA sample comprising a cDNA nucleotide sequence of a foreign gene of interest is prepared. The DNA sample is preferably one that can be confirmed as a single plasmid by electrophoresis at a concentration of no less than 25 ng/μl. The following explains an example using NotI site to insert a foreign gene into a DNA encoding a viral genomic RNA. In cases where NotI recognition sites are contained in a cDNA nucleotide sequence of interest, the nucleotide sequence should be altered using site-directed mutagenesis or the like, such that the encoded amino acid sequence does not change, and the NotI sites are preferably excised in advance. The gene fragment of interest is amplified from this sample by PCR, and then recovered. By adding a NotI site to the 5′ region of two primers, both ends of the amplified fragment become NotI sites. E-I-S sequences, or parts thereof, are included in the primers such that, after a foreign gene is inserted into the viral genome one E-I-S sequence is placed between the ORF of the foreign gene and the ORF of the viral genes on each side.

For example, to guarantee NotI cleavage, the forward synthetic DNA sequence has a form in which an arbitrary sequence of no less than two nucleotides (preferably four nucleotides not comprising a NotI recognition site-derived sequence such as GCG and GCC, and more preferably ACTT) is selected at the 5′-side, and a NotI recognition site gcggccgc is added to its 3′-side. To this 3′-side, nine arbitrary nucleotides, or nine plus a multiple of six nucleotides are further added as a spacer sequence. Further to the 3′-side, a sequence corresponding to about 25 nucleotides of the ORF of a desired cDNA including the initiation codon ATG is added. The 25 nucleotides are preferably selected from a desired cDNA such that the final nucleotide at the 3′-end of the forward synthetic oligo DNA is G or C.

For the reverse synthetic DNA sequence, no less than two arbitrary nucleotides (preferably four nucleotides not comprising a NotI recognition site-derived sequence such as GCG and GCC, and more preferably ACTT) are selected at the 5′-side, a NotI recognition site ‘gcggccgc’ is added to its 3′-side, and further an oligo DNA fragment is added to this 3′-side for length adjustment. The length of this oligo DNA is designed such that the chain length of the NotI fragment, the final PCR-amplification product, will become a multiple of six nucleotides including the added E-I-S sequences (so-called “rule of six”: Kolakofski, D., et al., J. Virol. 72: 891-899, 1998; Calain, P. and Roux, L., J. Virol. 67: 4822-4830, 1993; Calain, P. and Roux, L., J. Virol. 67: 4822-4830, 1993). When an E-I-S sequence is added to the 3′-side of the inserted oligo DNA fragment of this primer, a complementary strand sequence of the Sendai virus S sequence, preferably 5′-TTTCACCCT-3′ (SEQ ID NO: 9), 5′-TTTGACCCT-3′ (SEQ ID NO: 10), or 5′-ATTCACCCT-3′ (SEQ ID NO: 11), a complementary strand sequence of I sequence, preferably 5′-AAG-3′, a complementary strand sequence of E sequence, preferably 5′-TTTTTCTTACTACGG-3′ (SEQ ID NO: 12) are added. Further to this 3′-side, a complementary strand sequence with G or C as the last nucleotide is added, in which the length was selected to be about 25 nucleotides counted backwards from the termination codon of a desired cDNA sequence. Thus the 3′-end of the reverse synthetic DNA is made.

PCR can be performed by a standard method using Taq polymerase or other DNA polymerases. The amplified fragments of interest are digested with NotI and then inserted into the NotI site of a plasmid vector such as pBluescript™ (Stratagene). The nucleotide sequence of the PCR products thus obtained is confirmed with a sequencer, and plasmids comprising the correct sequence are selected. The inserted fragment is excised from these plasmids using NotI, and cloned into the NotI site of a plasmid comprising the genomic cDNA. The recombinant Sendai virus cDNA can also be obtained by inserting the fragment directly into the NotI site without using a plasmid vector.

For example, a recombinant Sendai virus genomic cDNA can be constructed according to the methods described in literature (Yu, D. et al., Genes Cells 2: 457-466, 1997; Hasan, M. K. et al., J. Gen. Virol. 78: 2813-2820, 1997). For example, an 18 bp spacer sequence (5′-(G)-CGGCCGCAGATCTTCACG-3′) (SEQ ID NO: 13) comprising a NotI restriction site is inserted between the leader sequence and the ORF of N protein in a cloned Sendai virus genomic cDNA (pSeV(+)) to obtain plasmid pSeV18⁺b(+), which comprises an autocleavage ribozyme site derived from an antigenomic strand of delta hepatitis virus (Hasan, M. K. et al., J. General Virology 78: 2813-2820, 1997). A recombinant Sendai virus cDNA comprising a desired foreign gene can be obtained by inserting a foreign gene fragment into the NotI site of pSeV18⁺b(+).

A vector of the present invention can be reconstructed by transcribing a DNA encoding the genomic RNA of a recombinant virus thus prepared in cells under the presence of the above-described viral proteins (L, P, and N). The present invention provides DNAs encoding the viral genomic RNAs of a vector of the present invention for producing a vector of this invention. The present invention also relates to the use of DNAs encoding the genomic RNAs of a vector for application in producing a vector of this invention. Recombinant viruses can be reconstructed by the methods known in literature (WO 97/16539; WO 97/16538; WO 00/70055; WO 00/70070; WO 01/18223; WO 03/025570; Durbin, A. P. et. al., Virology 235: 323-332, 1997; Whelan, S. P. et al., Proc. Natl. Acad. Sci. USA 92: 8388-8392, 1995; Schnell. M. J. et al., EMBO J. 13: 4195-4203, 1994; Radecke, F. et al., EMBO J. 14: 5773-5784, 1995; Lawson, N. D. et al., Proc. Natl. Acad. Sci. USA 92: 4477-4481, 1995; Garcin, D. et al., EMBO J. 14: 6087-6094, 1995; Kato, A. et al., Genes Cells 1: 569-579, 1996; Baron, M. D. and Barrett, T, J. Virol. 71: 1265-1271, 1997; Bridgen, A. and Elliott, R. M., Proc. Natl. Acad. Sci. USA 93: 15400-15404, 1996; Hasan, M. K. et al., J. Gen. Virol. 78: 2813-2820, 1997; Kato, A. et al., EMBO J. 16: 578-587, 1997; Yu, D. et al., Genes Cells 2: 457-466, 1997; Tokusumi, T. et al., Virus Res. 86: 33-38, 2002; Li, H.-O. et al., J. Virol. 74: 6564-6569, 2000). With these methods, minus strand RNA viruses including parainfluenza virus, vesicular stomatitis virus, rabies virus, measles virus, rinderpest virus, and Sendai virus can be reconstructed from DNAs. Vectors of the present invention can be reconstructed according to these methods. A viral vector DNA made defective of F gene, HN gene, and/or M gene does not form infectious virions. However, infectious virions can be formed by introducing the missing gene(s) and/or gene(s) encoding the envelope protein(s) of another virus into the host cell, and then expressing the gene(s).

Specifically, a virus can be produced according to the steps of: (a) transcribing cDNAs encoding the genomic RNAs (minus strand RNAs) of a minus strand RNA virus or complementary strands thereof (plus strands) in cells expressing N, P, and L proteins; and (b) collecting the RNP complex comprising the genomic RNA from these cells or culture supernatant thereof. For transcription, a DNA encoding a genomic RNA is linked to the downstream of an appropriate promoter. The genomic RNAs thus transcribed are replicated in the presence of N, L, and P proteins to form the RNP complex. Then, in the presence of M, HN, and F proteins, virions enclosed in an envelope are formed. For example, a DNA encoding a genomic RNA can be linked to the downstream of a T7 promoter and transcribed to RNA by a T7 RNA polymerase. A desired promoter beside those comprising a T7 polymerase recognition sequence can also be used. Alternatively, RNAs transcribed in vitro may be transfected into cells. N, L, and P proteins may be expressed in cells using appropriate expression vectors such as plasmids. Examples of promoters include a CMV promoter and a CAG promoter (Niwa, H. et al., Gene. 108: 193-199, 1991-Japanese Patent Application Kokai Publication No. (JP-A H3-168087).

Enzymes essential for the initial transcription of a genomic RNA from DNA, such as T7 RNA polymerase, can be supplied by introducing plasmid vectors or viral vectors that express them or by, for example, incorporating genes of these enzymes into the cellular chromosome to enable induction of their expression, and then inducing expression at the time of viral reconstruction. Further, the genomic RNA and viral proteins essential for the vector reconstruction are supplied, for example, by introducing plasmids that express them. In supplying these viral proteins, the helper virus of a wild type minus strand RNA virus or certain types of mutants thereof can be used, but is not preferred since contamination of these viruses may occur.

Methods of introducing DNAs that express genomic RNAs into cells include, for example, (i) methods to make DNA precipitates that can be taken in by the cells of interest; (ii) methods to make positively charged complexes comprising DNAs that have low cytotoxicity and are suitable for intake by the cells of interest; and (iii) methods using electric pulses to instantaneously bore pores on the cell membrane with sufficient sizes through which DNA molecules can pass into the cells of interest.

Various transfection reagents can be used in method (ii), for example, DOTMA (Roche), Superfect (QIAGEN #301305), DOTAP, DOPE, DOSPER (Roche #1811169), and such. An example of (i) is a transfection method using calcium phosphate, and although DNAs transferred into cells by this method are taken in phagosomes, a sufficient amount of DNA is also known to enter the nucleus (Graham, F. L. and Van Der Eb, J., Virology 52: 456, 1973; Wigler, M. and Silverstein, S., Cell 11: 223, 1977). Chen and Okayama investigated the optimization of transfer techniques, reporting that (1) conditions for cell incubation with co-precipitates are 2 to 4% CO₂, 35° C., and 15 to 24 hours, (2) circular DNA has a higher activity than linear DNA, and (3) optimal precipitation is obtained when the precipitate mixture has a DNA concentration of 20 to 30 μg/ml (Chen, C. and Okayama, H., Mol. Cell. Biol. 7: 2745, 1987). The methods of (ii) are suitable for transient transfections. Transfection methods where a DEAE-dextran (Sigma #D-9885 M.W. 5×10⁵) mixture is prepared based on a desired DNA concentration ratio have been known formerly. Since most complexes are decomposed in endosomes, chloroquine may also be added to enhance the efficiency (Calos, M. P., Proc. Natl. Acad. Sci. USA 80: 3015, 1983). The methods of (iii) are referred to as electroporation methods, and are more general ones than the methods (i) and (ii) because they are not cell selective. The efficiency of the methods is considered to be good under optimal conditions of: duration of pulse electric current, pulse shape, electric field potency (gap between electrodes, voltage), buffer conductivity, DNA concentration, and cell density.

Of the above three categories, the methods of (ii) are simple to operate and can examine many samples with a large amount of cells, and thus transfection reagents are suitable for introducing DNAs into cells in vector reconstruction. Transfection reagents such as Superfect Transfection Reagent (QIAGEN, Cat No. 301305), DOSPER Liposomal Transfection -Reagent (Roche, Cat No. 1811169), and TransIT (Mirus, Cat. No. MIR2300) can be preferably used. However it is not limited thereto.

Specifically, virus reconstruction from cDNA can be carried out, for example, as follows:

In a plastic plate of about 6- to 24-wells, or a 100-mm Petri dish or the like, simian kidney-derived LLC-MK2 cells are cultured till near 100% confluency in a minimum essential medium (MEM) containing 10% fetal calf serum (FCS) and antibiotics (100 units/ml penicillin G and 100 μg/ml streptomycin), and infected at 2 PFU/cell with, for example, the recombinant vaccinia virus vTF7-3, which expresses T7 RNA polymerase and has been inactivated by 20-minutes of UV irradiation in the presence of 1 μg/ml psoralen (Fuerst, T. R. et al., Proc. Natl. Acad. Sci. USA 83: 8122-8126, 1986; Kato, A. et al., Genes Cells 1: 569-579, 1996). The amount of psoralen added and the UV irradiation time can be adjusted appropriately. One hour after infection, 2 to 60 μg and more preferably 3 to 20 μg of DNA encoding the genomic RNA of a recombinant Sendai virus is transfected together with plasmids expressing the trans-acting viral proteins essential for viral RNP production (0.5 to 24 μg of pGEM-N, 0.25 to 12 μg of pGEM-P, and 0.5 to 24 μg of pGEM-L) (Kato, A. et al., Genes Cells 1: 569-579, 1996) by a lipofection method using Superfect (QIAGEN) or such. The amount ratio of the expression vectors encoding N, P, and L proteins is preferably 2:1:2; and the amount of plasmids was appropriately adjusted within a range of 1 to 4 μg of pGEM-N, 0.5 to 2 μg of pGEM-P, and 1 to 4 μg of pGEM-L, for example.

The transfected cells are cultured in serum-free MEM containing 100 μg/ml of rifampicin (Sigma) and cytosine arabinoside (AraC) (Sigma), more preferably 40 μg/ml of cytosine arabinoside (AraC) alone if desired. Optimal drug concentrations are set so as to minimize the cytotoxicity caused by the vaccinia virus, and to maximize the virus recovery rate (Kato, A. et al., Genes Cells 1: 569-579, 1996). After 48 to 72 hours of culture following transfection, cells are harvested and then disintegrated by freeze-thaw three times. LLC-MK2 cells are infected with the disintegrated materials that contain RNPs and further cultured. Alternatively, the culture supernatant is collected and added to a culture medium of LLC-MK2 cells for infection, followed by cell culturing. Transfection can be conducted by, for example, forming a complex with lipofectamine, polycationic liposome, or the like, and then introducing the complex into cells. Specifically, various transfection reagents can be used, for example, DOTMA (Roche), Superfect (QIAGEN #301305), DOTAP, DOPE, and DOSPER (Roche #1811169). Chloroquine may also be added to prevent decomposition in the endosome (Calos, M. P., Proc. Natl. Acad. Sci. USA 80: 3015, 1983). In the cells to which RNPs have been introduced, the vector is amplified as a result of the expression of viral genes from the RNP and RNP replication. By diluting the viral fluid (supernatant) thus obtained (for example, 10⁶-fold), and then repeating the amplification using new cells, the vaccinia virus vTF7-3 can be completely eliminated. Such amplification is repeated, for example, three or more times. The vectors thus obtained can be stored at −80° C. In order to reconstruct a viral vector incapable of transmission and defective of a gene(s) encoding an envelope protein(s), LLC-MK2 cells expressing the envelope protein(s) may be used for the transfection, or a plasmid(s) expressing the envelope protein(s) may be co-transfected. Alternatively, a defective viral vector can be amplified by culturing the transfected cells further overlaid with LLK-MK2 cells expressing the envelope protein(s) (see WO 00/70055 and WO 00/70070).

Titers of the virus thus recovered can be determined, for example, by measuring CIU (Cell Infectious Unit) or hemagglutination activity (HA) (WO 00/70070; Kato, A. et al., Genes Cells 1: 569-579, 1996; Yonemitsu, Y. & Kaneda, Y, Hemaggulutinating virus of Japan-liposome-mediated gene delivery to vascular cells. Ed. by Baker AH. Molecular Biology of Vascular Diseases, Method in Molecular Medicine: Humana Press: pp. 295-306, 1999). Titers of the vector carrying a GFP (green fluorescent protein) marker gene and the like can be quantified by directly counting the infected cells using the marker as an indicator (for example, as GFP-CIU). Titers thus measured can be handled in the same way as CIU (WO 00/70070).

As long as a viral vector can be reconstructed, the host cell used in the reconstruction is not particularly limited. For example, in reconstructing Sendai viral vectors and such, cultured cells such as LLC-MK2 cells (ATCC CCL-7) and CV-1 cells (for example ATCC CCL-70) derived from monkey kidney, BHK cells derived from hamster kidney (for example ATCC CCL-10), and cells derived from humans can be used. By expressing a suitable envelope protein(s) in these cells, infectious virions that contain the protein(s) in their envelope can also be obtained. Further, to obtain large quantities of a Sendai viral vector, a viral vector obtained from an above-described host can be used to infect embryonated hen eggs for amplification of the vector. The method for generating viral vectors using hen eggs has already been developed (Nakanishi, M. et al. ed. “State-of-the-Art Technology Protocol in Neuroscience Research 111 (Shinkei Kagaku Kenkyu-no Sentan Gijutu Protocol III); Molecular Neuron Physiology (Bunshi Sinkeisaibou Seirigaku)”, Koseisha, Osaka, pp. 153-172, 1993). Specifically, for example, an embryo is grown by placing a fertilized egg in an incubator, and culturing for nine to twelve days at 37 to 38° C. After a viral vector is inoculated into the allantoic cavity, the egg is cultured for several days (for example, three days) to allow the viral vector to proliferate. Conditions such as the incubation period may vary depending upon the recombinant Sendai virus being amplified. Then, the allantoic fluid comprising the vector is collected. The Sendai viral vector can be separated and purified from the allantoic fluid according to a standard method (Tashiro, M., “Virus Experiment Protocol”, Nagai, Ishihama, ed., Medical View Co., Ltd., pp. 68-73, 1995).

For example, reconstruction and production of Sendai virus vectors defective of F gene can be performed as described below (see WO 00/70055 and WO 00/70070):

<1> Construction of F-Gene Defective Sendai Virus Genomic cDNA and F Gene-Expressing Plasmids

A full-length genomic cDNA of Sendai virus (SeV), pSeV18⁺b(+) cDNA (Hasan, M. K. et al., J. General Virology 78: 2813-2820, 1997) (“pSeV18⁺b(+)” is also referred to as “pSeV18⁺”), is digested with SphI/KpnI to generate a fragment (14673 bp). This fragment is then cloned into pUC18 to prepare a plasmid pUC18/KS, on which an F gene-deficient site is constructed. F gene deletion is created by a combination of PCR and ligation methods. As a result, the F gene ORF (ATG−TGA= 1698 bp) is removed. Then, atgcatgccggcagatga (SEQ ID NO: 14), for example, is ligated to construct an F gene-defective type SeV genomic cDNA (pSeV18⁺/ΔF). A PCR product formed using primers for the upstream region of F [forward: 5′-gttgagtactgcaagagc/SEQ ID NO: 15, reverse: 5′-tttgccggcatgcatgtttcccaaggggagagttttgcaacc/SEQ ID NO: 16], and a PCR product formed using primers for the downstream region of F [forward: 5′-atgcatgccggcagatga/SEQ ID NO: 17, reverse: 5-tgggtgaatgagagaatcagc/SEQ ID NO: 18] were ligated via the EcoT22I restriction site. A 4931 bp fragment that covers the region comprising the F gene-defective site is recovered by digesting the plasmid thus obtained with SacI and SalI, and cloned into pUC18 to form pUC18/dFSS. This pUC18/dFSS is digested with DraIII, and the fragment recovered is used to replace the DraIII fragment that covers the region comprising the F gene of pSeV18⁺ through ligation to obtain the plasmid pSeV18⁺/ΔF.

A foreign gene is inserted, for example, into the NsiI and NgoMIV restriction enzyme sites in the F gene-deleted site of pUC18/dFSS. For this, a foreign gene fragment may be, for example, amplified using an NsiI-tailed primer and an NgoMIV-tailed primer.

### <2> Production of the Helper Cell Expressing SeV-F Protein

To construct a Cre/loxP-induced expression plasmid expressing Sendai virus F gene (SeV-F), SeV-F gene is amplified by PCR and inserted into an unique SwaI site of a plasmid pCALNdlw (Arai, T. et al., J. Virology 72: 1115-1121, 1998), which is designed to enable expression of a gene product by Cre DNA recombinase, to construct a pCALNdLw/F plasmid.

To recover infectious virions from F gene-defective genome, a helper cell line expressing SeV-F protein is established. For example, LLC-MK2 cells derived from monkey kidney, which is commonly used for SeV proliferation, can be used. LLC-MK2 cells are cultured in MEM supplemented with 10% heat-inactivated fetal bovine serum (FBS), penicillin G sodium (50 units/ml), and streptomycin (50 μg/ml) at 37° C. in 5% CO₂. Since the SeV-F gene product is cytotoxic, LLC-MK2 cells are transfected with the above-described pCALNdLw/F plasmid, which is designed to express the F gene product with Cre DNA recombinase, by a calcium phosphate method using a mammalian transfection kit (Stratagene) according to a protocol publicly well known.

The pCALNdLw/F plasmid (10 μg) is introduced into LLC-MK2 cells grown to 40% confluence in a 10-cm plate, and the cells are then cultured in 10% FBS/MEM (10 ml) in a 5% CO₂ incubator at 37° C. for 24 hours. Then, the cells are detached and suspended in the medium (10 ml). The suspension is then seeded on five 10-cm dishes: 5 ml for one dish, 2 ml each for two dishes, and 0.2 ml each for two dishes. The cells are cultured in MEM (10 ml) containing G418 (GIBCO-BRL) (1200 μg/ml) and 10% FBS for 14 days, and the medium is exchanged every two days. Cell lines stably transduced the gene are selected. The cells growing on the above medium are resistant against G418, and then recovered using a cloning ring. Each cell clone thus recovered continues to be cultured in 10-cm plates until it becomes confluent.

After the cells have grown to confluence in 6-cm dishes, F protein expression is induced by infecting the cells with Adenovirus AxCANCre, for example, at MOI=3, according to the method of Saito, et al. (Saito et al., Nucl. Acids Res. 23: 3816-3821, 1995; Arai, T. et al., J. Virol 72: 1115-1121, 1998).

### <3> Reconstruction and Amplification of F Gene-Defective Sendai Virus (SeV/ΔF)

The above-described pSeV18⁺/ΔF plasmid, into which a foreign gene has been inserted, is used to transfect LLC-MK2 cells as follows. LLC-MK2 cells are seeded at 5×10⁶ cells/dish in 100-mm dishes. When genomic RNA transcription is carried out with T7 RNA polymerase, the cells are cultured for 24 hours, and then infected with a recombinant vaccinia virus expressing T7 RNA polymerase at MOI= 2 or such, for one hour at room temperature, wherein the virus has been treated with psoralen and long-wave ultraviolet rays (365 nm) for 20 minutes (PLWUV-VacT7: Fuerst, T. R. et al., Proc. Natl. Acad. Sci. USA 83: 8122-8126, 1986). For the ultraviolet ray irradiation of the vaccinia virus, for example, an UV Stratalinker™ 2400 equipped with five 15-watt bulbs can be used (Catalogue No. 400676 (100V), Stratagene, La Jolla, Calif., USA). The cells are washed with serum-free MEM, and then transfected with a plasmid expressing the genomic RNA as well as expression plasmids expressing N, P, L, and F plus HN proteins of Paramyxovirus using an appropriate lipofection reagent. The amounts of these plasmids can be preferably set to 6:2:1:2:2 in this order, without being limited thereto. For example, a genomic RNA-expressing plasmid as well as expression plasmids expressing N, P, L, and F plus HN proteins (pGEM/NP, pGEM/P, pGEM/L, and pGEM/F-HN; WO 00/70070, Kato, A. et al., Genes Cells 1: 569-579, 1996) are transfected at a respective amount of 12, 4, 2, 4 and 4 μg per dish. After culturing for several hours, the cells are washed twice with serum-free MEM, and cultured in MEM containing 40 μg/ml of cytosine β-D-arabinofuranoside (AraC: Sigma, St. Louis, Mo.) and 7.5 μg/ml of trypsin (Gibco-BRL, Rockville, Md.). These cells are recovered, and the pellets are suspended in Opti-MEM (10⁷ cells/ml). The suspensions are freeze-thawed three times, mixed with a lipofection reagent DOSPER (Boehringer Mannheim) (10⁶ cells/25 μl DOSPER), left at room temperature for 15 minutes, and used to transfect the above-described cloned F-expressing helper cells (10⁶ cells/well in a 12-well-plate), which were cultured in serum-free MEM (containing 40 μg/ml AraC and 7.5 μg/ml trypsin) to recover supernatants. Any virus defective of a gene other than F, for example, HN and/or M gene, can also be produced by methods similar to this.

In producing viral gene-defective vectors, for example, if two or more types of vectors, in which a different viral gene has been deleted from the viral genome, are introduced into the same cell, the viral protein defective of the each vector is supplied through the expression of the other vector and thus such mutual complementation allows the construction of infectious virions and the amplification of the viral vectors through the replication cycle. As a result, mixtures of the individual viral gene-defective virus vectors can be produced at a low cost and on a large scale. Since such viruses lack any viral gene(s), their genome size is smaller than that of the intact virus. Thus, such viruses can retain larger foreign genes. In addition, such viruses are advantageous in that their release into the environment is under a control because such viruses, non-transmissible based on the defectiveness of the viral gene(s), are simply diluted following once released to the outside of the cell and become sterile due to the difficulties in maintaining infection. For example, vectors encoding Aβ and Th2 cytokine may be constructed separately so as to complement each other, and may be used for co-infection. The present invention provides compositions that comprise a minus strand RNA viral vector encoding a polypeptide comprising Aβ, and a minus strand RNA viral vector encoding a Th2 cytokine. The present invention also provides kits that comprise a minus strand RNA viral vector encoding a polypeptide comprising Aβ, and a minus strand RNA viral vector encoding a Th2 cytokine. Such compositions and kits can be used to reduce amyloid βdeposition.

After a transmissible minus strand RNA viral vector is administered to an individual or cell, and when the proliferation of the viral vector needs to be arrested upon, for example, the completion of the treatment, it is also possible to specifically arrest only the proliferation of the viral vector without incurring any damage to the host by administering an RNA-dependent RNA polymerase inhibitor.

According to the methods of the present invention, a viral vector of this invention can be released into the culture medium of the virus-producing cells, for example, at a titer of 1×10⁵ CIU/ml or more, preferably 1×10⁶ CIU/ml or more, more preferably 5×10⁶ CIU/ml or more, more preferably 1×10⁷ CIU/ml or more, more preferably 5×10⁷ CIU/ml or more, more preferably 1×10⁸ CIU/ml or more, and more preferably 5×10⁸ CIU/ml or more. Viral titers can be measured by methods disclosed in the present specification and others (Kiyotani, K. et al., Virology 177(1): 65-74, 1990; WO 00/70070).

The recovered viral vectors can be purified to become substantially pure. Purification of the vectors can be performed by purification and separation methods publicly known in literature, including filtration, centrifugal separation, and column purification, or any combinations thereof. “Substantially pure” means what a viral vector accounts for a major proportion of a sample in which the viral vector exists as a component. Typically, a substantially pure viral vector can be identified by confirming that the proportion of proteins derived from the viral vector is 10% or more of all the proteins in a sample, preferably 20% or more, more preferably 50% or more, preferably 70% or more, more preferably 80% or more, and even more preferably 90% or more (excluding, however, proteins added as carriers and stabilizers). Examples of specific methods for purifying viruses are those that use cellulose sulfate ester or cross-linked polysaccharide sulfate ester (Japanese Patent Application Kokoku Publication No. (JP-B) S-62-30752, JP-B S-62-33879, and JP-B S-62-30753), and methods that attach viruses to polysaccharides comprising fucose sulfate and/or degradation products thereof (WO 97/32010).

In preparing compositions comprising a vector, the vector can be combined with a desired pharmaceutically acceptable carrier or vehicle, as necessary. A “pharmaceutically acceptable carrier or vehicle” refers to a material that can be administered together with a vector without significantly inhibiting the vector-mediated gene introduction. For example, a vector can be appropriately diluted with a physiological saline or phosphate-buffered saline (PBS) to form a composition. When a vector is grown in hen eggs or the like, the “pharmaceutically acceptable carrier or vehicle” may comprise allantoic fluids. Further, compositions comprising a vector may include carriers or vehicles such as deionized water and 5% dextrose aqueous solution. Furthermore, compositions may comprise, besides the above, plant oils, suspending agents, surfactants, stabilizers, biocides, and so on. The compositions can also comprise preservatives or other additives. The compositions comprising vectors of the present invention are useful as reagents and pharmaceuticals. The present invention relates to methods for producing anti-amyloid deposition agents, which comprise the step of producing a composition comprising a minus strand RNA viral vector encoding an Aβ peptide or cells to which the vector has been introduced, and a pharmaceutically acceptable carrier or vehicle. The present invention also relates to the use of minus strand RNA viral vectors, cells producing the vectors, or cells to which the vectors have been introduced, in producing anti-amyloid deposition agents.

Compositions comprising a vector of the present invention may contain in combination as a carrier, organic materials such as biopolymer and inorganic materials such as hydroxyapatite, specifically, collagen matrix, polylactate polymer or copolymer, polyethylene glycol polymer or copolymer, and chemical derivatives thereof.

The compositions of the present invention may contain a Th2 cytokine and/or a nucleic acid encoding a Th2 cytokine in an expressible manner. Such a Th2 cytokine may be the full length (wild-type) polypeptide or a partial peptide as long as it retains the activity. For example, deletion of N-terminal and/or C-terminal amino acids (for example, 1 to 30 amino acids, specifically, 1, 2, 3, 4, 5, 10, 15, 20, or 25 amino acids) does not likely affect cytokine activity. Furthermore, the N-terminal signal sequence can be appropriately replaced by another signal sequence. Alternatively, the cytokine may be expressed as a fused protein with other peptides. The nucleic acid encoding a Th2 cytokine may be inserted in an expression vector, or connected to an appropriate promoter, such as actin promoter, EF1 promoter, CMV promoter, CAG promoter, and a like. The nucleic acid may be contained in a minus strand RNA viral vector or any other expression vectors. When a Th2 cytokine is encoded in a minus strand RNA viral vector, the Th2 cytokine may be encoded in the same vector as Aβ, alternatively, the Th2 cytokine may be encoded in another minus strand RNA viral vector. Specifically, when an Aβ-encoding minus strand RNA viral vector of the present invention does not encode a Th2 cytokine, Th1/Th2 balance can be shifted to Th2 by co-administering a Th2 cytokine or a Th2 cytokine vector in combination with the Aβ-encoding vector of the invention. For example, the Th2 cytokines that can be used are IL-4, IL-5, IL-6, IL-9, IL-10, and IL-13, or a combination thereof. The Th2 cytokine is preferably selected from a group consisting of IL-4, IL-10, and IL-13.

Compositions of the present invention may also comprise an adjuvant. Specifically, even when a vector of the present invention does not encode a Th2 cytokine, Th1/Th2 balance can be shifted to Th2 using a composition comprising a Th2 adjuvant. A Th2 adjuvant refers to an adjuvant that predominantly activates type 2 helper T cells (Th2 cells) over type 1 helper T cells (Th1 cells). Specifically, the adjuvant includes aluminum hydroxide (alum), cholera toxin (B subunit), protein extract from Schistosoma mansoni eggs (for example, Lacto-N-fucopentaose III), and so on (Grun, J. L. and R H. Maurer, Cellular Immunol. 121: 134-145, 1989; Holmgren, J. et al., Vaccine 11:1179-1184, 1993; Wilson, A. D. et al., Vaccine 11: 113-118, 1993; Lindsay, D. S. et al., Int. Arch. Allergy Immunol. 105: 281-288, 1994; Xu-Amano, J. et al., J. Exp. Med. 178: 1309-1320, 1993; Okano, M. et al., J. Immunol. 167(1): 442-50, 2001). Compositions of the present invention may contain any other ingredients. For example, they may contain a marker for monitoring administration, or one or more compounds such as immune system-regulators, signal transduction regulators, cytokines, hormones, receptors, antibodies, and fragments thereof. Compositions of the present invention may contain one or more vectors encoding one or more polypeptides such as signal transduction regulators, signal transduction regulators, cytokines, hormones, receptors, antibodies, and fragments thereof.

Administration of a minus strand RNA virus thus generated or a composition comprising the virus to individuals induces an immune response to Aβ and thereby reduces Aβ deposition. The vector may be administered in combination with a Th2 cytokine, a Th2 cytokine-expressing vector, or a Th2 adjuvant. The vector administration may be an in vivo administration or a cell-mediated ex vivo administration. The minus strand RNA viral vector used in inoculation does not need to be infectious virions, and may be non-infectious virions, virus core (an RNP complex comprising the genome and genome-binding viral proteins), or the like, as long as it has the ability to express antigen polypeptide genes carried by the genome RNA. Herein, the minus strand RNA viral vector refers to a complex that comprises a ribonucleoprotein (RNP) complex containing a genome RNA derived from a minus strand RNA virus and viral proteins required for replication of the genome RNA and expression of the genes carried by the virus, wherein the complex replicates the genome RNA in the infected cell and expresses the carried genes. The RNP is, for example, a complex comprising genome RNAs and N, L, and P proteins of a minus strand RNA virus. Specifically, minus strand RNA viral vectors of the present invention include infectious virions, non-infectious virions (also referred to as virus-like particles (VLPs)), and RNPs comprising the genome RNAs and viral proteins that bind to the genome RNA such as nucleocapsids of a minus strand RNA virus. When introduced into cells, even RNP (viral core) in which the envelope is removed from the virion can replicate the viral genome RNA in the cells (WO 97/16538; WO 00/70055). RNPs or VLPs are desirably used for inoculation, for example, in combination with a transfection reagent or the like (WO 00/70055; WO 00/70070).

When a vector inoculation is via cells, a minus strand RNA viral vector is introduced into appropriate culture cells, or cells collected from an animal subject of the inoculation, or such. When infecting cells with a minus strand RNA virus in vitro (for example, in a test tube or dish), the infection can be performed, for example, in vitro (or ex vivo) in a desired physiological solution, such as a culture medium or physiological saline. MOI (multiplicity of infection; i.e., the number of infected viruses per cell) preferably ranges from 1 to 1000, more preferably from 2 to 500, even more preferably from 3 to 300, still more preferably from 5 to 100. Only a short period of contact between a minus strand RNA virus and cells is sufficient. The contact may be, for example, 1 minute or longer, preferably 3 minutes or longer, 5 minutes or longer, 10 minutes or longer, or 20 minutes or longer, such as 1 to 60 minutes, more specifically about 5 to 30 minutes. Alternatively, the contact may take place over a longer period of time, for example, several days or longer.

RNPs or non-infectious virions (virus-like particles (VLPs)) comprising a viral genome RNA can be introduced into cells by conventional transfection methods. Specifically, transfection to cells can be achieved by various techniques known to those skilled in the art, such as calcium phosphate methods (Chen, C. & Okayama, H., BioTechniques 6:632-638, 1988; Chen, C. and Okayama, H., Mol. Cell. Biol. 7: 2745, 1987), DEAE-dextran methods (Rosenthal, N., Methods Enzymol. 152:704-709, 1987), various liposome-based transfection reagents (Sambrook, J. et al., Molecular Cloning: A Laboratory Manual, Cold Spring Harbor Laboratory, Cold Spring Harbor, N.Y., 1989), and electroporation methods (Ausubel, F. et al., In Current Protocols in Molecular Biology, John Wiley and Sons, NY, Vol. 1, Ch. 5 and 9, 1994). Chloroquine may be added in the transfection to suppress endosomal degradation (Calos, M. P., Proc. Natl. Acad. Sci. USA 80: 3015, 1983). Transfection reagents include DOTMA (Roche), Superfect Transfection Reagent (QIAGEN, Cat No. 301305), DOTAP, DOPE, DOSPER (Roche #1811169), TransIT-LT1 (Mirus, Product No. MIR 2300), CalPhos™ Mammalian Transfection Kit (Clontech #K2051-1), CLONfectin™ (Clontech #8020-1) and such. Enveloped viruses are known to take up host cell-derived proteins during viral particle formation. Such proteins may have some antigenicity and cause cytotoxicity when introduced into cells (J. Biol. Chem., 272: 16578-16584, 1997). Thus, it is advantageous-to-use-envelope free RNPs (WO 00/70055).

Viral RNPs can be formed directly in cells by introducing an expression vector of a viral genome RNA and expression vectors encoding viral proteins (N, P, and L proteins) required for replication of the genome RNA into cells. Cells to which viral vectors have been introduced may be prepared by such a procedure.

Once prepared, the cells to which a minus strand RNA viral vector has been introduced may be cultured for about 12 hours to 5 days (preferably for 1 to 3 days) to express an Aβ peptide. The harvested cells can be used directly or as cell lysates to inoculate to animals; however, Aβ peptide-expressing cells are preferably used for inoculation. Aβ having a signal peptide may be expressed from the vector and secreted to the outside of the cell. Lysates of cells introduced a vector can be prepared by a procedure that lyses the cell membrane with a detergent or a procedure that involves repetition of freeze-thaw cycles. Non-ionic detergents, such as Triton X-100 and Nonidet P-40, can be applied within the concentration range of 0.1 to 1%. Such lysates can be obtained by, for example, collecting cell cluster with centrifugation after PBS washing and re-suspension in TNE buffer [25 mM Tris-HCl (pH7.5), 150 mM NaCl, 1 mM EDTA, 1% Nonidet P-40], and incubating the cell suspension allowed to stand on ice for 10 to 30 minutes. If the protein to be used as an antigen is soluble in the cytoplasm, the prepared lysates can be centrifuged (10,000×g for 10 minutes) to remove the unnecessary insoluble fraction as precipitates and the resulting supernatant can be used for immunization. If a lysate is administered at a site where detergents are undesirable, the lysate may be prepared by re-suspending the cells in PBS after washing and disrupting the cells through 5 to 6 freeze-thaw cycles. The lysate may be administered in combination with a transfection reagent.

Alternatively, immunity can be induced by administering nucleic acids which produce an Aβ peptide-encoding minus strand RNA viral vector to an animal, and producing the Aβ peptide expressed by the minus strand RNA viral vector in the body of the animal. The nucleic acids generating a minus strand RNA viral vector refer to a set of nucleic acids that allow the expression of RNAs as well as the expression of a group of proteins required for the production of the recombinant minus strand RNA viral vector. Specifically, the nucleic acids refer to a set of nucleic acids comprising a nucleic acid encoding the genome RNA of an Aβ-encoding minus strand virus or a complementary strand thereof (antigenome RNA; i.e., plus strand), as well as nucleic acids encoding a group of viral proteins that construct an RNP comprising the genome RNA of the minus strand RNA virus. Such nucleic acids comprise an appropriate promoter to express the encoded RNAs or proteins. Such promoters include, for example, CMV promoters (Foecking, M. K, and Hofstetter H., Gene 45: 101-105, 1986), retroviral LTRs (Shinnik, T. M. et al., Nature, 293: 543-548, 1981), EF1 promoters, and CAG promoters (Niwa, H. et al., Gene. 108: 193-199, 1991. JP-A No. H3-168087). Plasmid vectors can be suitably used as nucleic acids. The genome of a minus strand RNA virus preferably carries the genes encoding a group of viral proteins (typically N, L, and P) that construct the RNP comprising the genome RNA, as well as all the genes of envelope-constituting proteins of the parental wild-type virus. If a minus strand RNA virus genome lacks any gene(s) of the envelope-constituting proteins, the nucleic acids encoding the envelope protein(s) (for example, the envelope protein(s) lacked by the genome, or other envelope protein(s), such as VSV-G) that complement the formation of infectious virions may be included in the set of nucleic acids described above. Administration of these nucleic acids to an animal results in the production of the recombinant minus strand RNA virus in the animal. A group of viral proteins that construct an RNP comprising the genome RNA of a minus strand RNA virus refer to the proteins that form an RNP together with the viral genome RNA, and constitute the nucleocapsid. These are a group of proteins required for genome replication and gene expression, and typically include N, P, and L proteins. Specifically, the present invention also relates to methods comprising the step of inoculating (i) a nucleic acid encoding the genome RNA of an Aβ-encoding minus strand RNA virus or a complementary strand thereof; and (ii) nucleic acids encoding proteins N, P, and L to an animal. In this animal, an RNP comprising the genome RNA is formed and an Aβ peptide is expressed. The genome may further encode a Th2 cytokine.

Alternatively, inoculation of cells or lysates thereof, to which a nucleic acid encoding the genome RNA or a complementary strand thereof and a group of viral proteins constituting the above-described RNP are introduced previously, may also be performed. See the following documents for more detailed information on the nucleic acids generating a minus strand RNA viral vector:

WO 97/16539; WO 97/16538; WO 00/70055; WO 00/70070; WO 01/18223; WO 03/025570; Durbin, A. P. et al., Virology 235: 323-332, 1997; Whelan, S. P. et al., Proc. Natl. Acad. Sci. USA 92: 8388-8392, 1995; Schnell. M. J. et al., EMBO J. 13: 4195-4203, 1994; Radecke, F. et al., EMBO J. 14: 5773-5784, 1995; Lawson, N. D. et al., Proc. Natl. Acad. Sci. USA 92: 4477-4481, 1995; Garcin, D. et al., EMBO J. 14: 6087-6094, 1995; Kato, A. et al., Genes Cells 1: 569-579, 1996; Baron, M. D. and Barrett, T., J. Virol. 71: 1265-1271, 1997; Bridgen, A. and Elliott, R. M., Proc. Natl. Acad. Sci. USA 93: 15400-15404, 1996; Hasan, M. K. et al., J. Gen. Virol. 78: 2813-2820, 1997; Kato, A. et al., EMBO J. 16: 578-587, 1997; Yu, D. et al., Genes Cells 2: 457-466, 1997; Tokusumi, T. et al., Virus Res. 2002: 86; 33-38, 1997; Li, H.-O. et al., J. Virol., 74: 6564-6569, 2000.

When an animal is inoculated with nucleic acids generating a minus strand RNA viral vector, a nucleic acid encoding the genome RNA of an Aβ-encoding minus strand RNA virus or a complementary strand thereof, and nucleic acids encoding N, P, and L proteins may be introduced into the animal at a weight ratio of 5:0.5:0.5:2. The nucleic acid ratio may be adjusted suitably. For example, when expression plasmids are used, the administration can be carried out using 5 to 1000 μg of a plasmid encoding an Aβ-encoding viral genome RNA (plus or minus strand), and 0.5 to 200 μg of an N-expressing plasmid, 0.5 to 200 μg of a P-expressing plasmid, and 2 to 500 μg of an L-expressing plasmid. Administration of the nucleic acids can be performed, for example, by injecting naked DNAs directly or after being combined with a transfection reagent. The transfection reagent includes, for example, lipofectamine and polycationic liposomes. Specifically, DOTMA (Roche), Superfect (QIAGEN #301305), DOTAP, DOPE, DOSPER (Roche #1811169), TransIT-LT1 (Minis, Product No. MIR 2300), or such can be used.

The dosage of a vector of the present invention to be administered varies depending on the type of disease, body weight, age, sex and symptoms of a patient, purpose of administration, dosage form in which a composition is introduced, method of administration, type of gene to be introduced, and so on. One skilled in the art can determine the appropriate dosage. The administration route can be suitably selected, and include transdermal, intranasal, transbronchial, intramuscular, intraperitoneal, intravenous, and subcutaneous routes, without being limited thereto. The particularly preferred routes are intramuscular injection (for example, into the gastrocnemial muscle), subcutaneous injection, intranasal dropping or spray, intradermal injection into palm or sole, direct injection into the spleen, intraperitoneal injection, and such. There may be one or more inoculation sites, for example, 2 to 15 sites. The inoculation dosage may be suitably adjusted depending on the animal subject, inoculation site, and inoculation frequency, and the like. It is preferable to administer the vector in combination with a pharmaceutically acceptable carrier at a dose within a range of about 10⁵ to about 10¹¹ CIU/ml, more preferably about 10⁷ to about 10⁹ CIU/ml, most preferably about 1×10⁸ to about 5×10⁸ CIU/ml. In terms of the virus titer, a single dose for human ranges from 1×10⁴ CIU to 5×10¹¹ CIU (cell infectious unit), and preferably from 2×10⁵ CIU to 2×10¹⁰ CIU. The frequency of administration is once, or several times as long as the side effects are within a clinically acceptable range. The number of administrations per day may also be determined in the same manner. Although a single dose produces significant effects, stronger effects can be obtained by introducing a vector more than once.

When a vector is administered more than once, for example, the second administration may be carried out approximately one year after the first administration. The preferred route of administration is, for example, intranasal or intramuscular administration. The administration may be repeated for stronger effects. Inoculation of the above-mentioned minus strand RNA viral vector carrying an Aft-encoding nucleic acid, nucleic acids generating the viral vector, cells introduced with the viral vector or the nucleic acids generating the vector, or lysates of the cells may be preferably used for multiple-dose administration.

When a vector is inoculated via cells (ex vivo administration), for example, human cells, and preferably autologous cells are infected with the minus strand RNA viral vector, and 10⁴ to 10⁹ cells, and preferably 10⁵ to 10⁸ cells, or lysates thereof, can be used. The vector can also be used to inoculate to non-human animals, and the dosage of administration can be converted from the above-described dosage based on the weight or volume ratio of the target site of administration (for example, an average value) of a subject animal to that of human. The subject for administration of the compositions comprising a vector of the present invention includes desired vertebrates (humans and non-human vertebrates) having an immune system, preferably birds and mammals, more preferably mammals (including humans and non-human mammals). Specifically, the mammals include humans and non-human primates such as monkeys, rodents such as mice and rats, and all other mammals as rabbits, goats, sheep, pigs, cattle, and dogs. The target animals for administration include, for example, individuals with Alzheimer's disease, individuals with an elevated level of Aβ, individuals with enhanced Aβ deposition, individuals carrying an Alzheimer-type mutant gene, and so on.

Immune responses against Aβ are induced by the methods of the present invention. Immune responses against Aβ can be confirmed by anti-Aβ antibody production, decrease of the amount of Aβ in brain tissues, and decrease in Aβ deposition, or the like. The production of anti-Aβ antibody can be evaluated by detection of the anti-Aβ antibody in blood samples. The antibody level can be measured with an ELISA (enzyme-linked immunosorbent assay) or Ouchterlony's method. An ELISA method is carried out, for example, by attaching an antigen to a microplate, preparing an antiserum and making a series of 2-fold dilutions of the prepared antiserum (starting solution 1:1000), and adding the diluted antisurum to the plate for antigen-antibody reaction to take place. Then, for color development, the antibody of the immunized animal is reacted with a xenogenic antibody, which has been enzymatically labeled with peroxidase and serves as a secondary antibody. The antibody titer can be calculated based on the dilution fold of the antibody when the absorbance is one half of the maximum color absorbance. Alternatively, in Ouchterlony's method, antigens and antibodies diffuse within an agar gel, and white precipitation lines are formed as a result of immunoprecipitation reactions. The precipitation lines can be used to determine the antibody titer, which is the dilution fold of the antiserum when the immunoprecipitation reaction occurs. Aβ level in the brain tissue can be determined, for example, using brain tissue extracts and a BioSource ELISA kit or the like.

The effect of reducing Aβ deposition (senile plaques) can be determined, for example, by the following procedure: treating brain tissue sections with 70% formic acid and after inactivating endogenous peroxidase with 5% H₂O₂, reacting the sections with an anti-Aβ antibody and performing DAB stain using a peroxidase-labeled secondary antibody. After the staining, the area of Aβ accumulated site can be determined by microscopic observation. If the area fraction of the accumulated site decreased in comparison with that when a vector of the present invention was not administered, the level of Aβ deposition can be judged as reduced. Alternatively, senile plaques in a living subject can be observed using MRI after intravenous administration of such a compound as 1-fluoro-2,5-bis-(3-hydroxycarbonyl-4-hydroxy) styrylbenzene (FSB), which has an affinity to amyloid or such (Higuchi M et al., Nat. Neurosci. 8(4):527-33, 2005; Sato, K. et al., Eur. J. Med. Chem. 39: 573, 2004; Klunk, W. E. et al., Ann. Neurol. 55(3):306-19, 2004). Effects of a vector of the present invention can be confirmed by such a non-invasive imaging technique for amyloids.

The present invention also relates to methods of determining immune responses to amyloid β, which comprise steps of: introducing a vector of the present invention or a composition comprising the vector into a subject with amyloid β deposition and of detecting anti-amyloid β antibody in the subject. The present invention also relates to methods of measuring amyloid β deposition, which comprise steps of: administering a vector of the present invention or a composition comprising the vector to a subject with amyloid β deposition and of detecting the level of amyloid β deposition in the subject. These methods allow immune responses to amyloid β and/or effects of reducing amyloid β deposition to be monitored.

### BRIEF DESCRIPTION OF THE DRAWINGS

FIG. 1 is a diagram showing the process of constructing Litmus SalI/NheIfrg-MtsHNtsPLmut-dF mIL10 (hereinafter abbreviated as Litmus TS-dF mIL10). Mutations were introduced into the F gene-deleted site of Litmus TS-dF to create a NotI site, into which the mIL-10 gene excised using NotI was inserted.

FIG. 2 is a diagram showing the process of constructing pSeV18+Aβ/MtsHNtsP511Lmut-dF (hereinafter abbreviated as pSeV18+Aβ/TS-dF). The Aβ gene, inserted into the NotI site of pBluescript™, was excised and inserted into the NotI site of pSeV18+NotI/TS-dF.

FIG. 3 is a diagram showing the process of constructing pSeV18+Aβ/MtsHNtsP511Lmut-dF mIL10 (hereinafter abbreviated as pSeV18+Aβ/TS-dF mIL10). The necessary fragments were excised from pSeV18+Aβ/TS-dF and Litmus TS-dF mIL10 using SalI and NheI, and switched.

FIG. 4 is a graph showing the level of serum anti-Aβ antibody of APP transgenic mice 4 and 8 weeks after the intranasal administration of SeV-Aβ1-43/mIL-10 or the control vector.

FIG. 5 is a photograph showing senile plaques in the frontal lobe of APP transgenic mice 8 weeks after the intranasal administration of SeV-Aβ1-43/mIL-10 or the control vector.

FIG. 6 is a photograph showing senile plaques in the parietal lobe of APP transgenic mice 8 weeks after the intranasal administration of SeV-Aβ1-43/mIL-10 or the control vector.

FIG. 7 is a photograph showing senile plaques in the hippocampus of APP transgenic mice 8 weeks after the intranasal administration of SeV-Aβ1-43/mIL-10 or the control vector.

FIG. 8 is a graph showing the result of quantifying senile plaques in APP transgenic mice (frontal lobe, parietal lobe, and hippocampus) 8 weeks after the intranasal administration of SeV-Aβ1-43/mIL-10 or the control vector.

FIG. 9 shows the result of examining lymphocyte infiltration in the central nervous system in APP transgenic mice 8 weeks after the intranasal administration of SeV-Aβ1-43/mIL-10 or the control vector. T cell infiltration in the frontal lobe was detected using CD3 as an indicator. Microglial alteration in the hippocampus was detected using Iba-1 as an indicator.

FIG. 10 is a set of graphs showing the amount of Aβ in the brain tissue of APP transgenic mice 8 weeks after the intranasal administration of SeV-Aβ1-43/mIL-10 or the control vector.

FIG. 11 is a graph showing the level of serum Aβ antibody of APP transgenic mice 4 and 8 weeks after the intramuscular administration of SeV-Aβ1-43/mIL-10 or the control vector.

FIG. 12 is a photograph showing senile plaques in the frontal lobe of APP transgenic mice 8 weeks after the intramuscular administration of SeV-Aβ1-43/mIL-10 or the control vector.

FIG. 13 is a photograph showing senile plaques in the parietal lobe of APP transgenic mice 8 weeks after the intramuscular administration of SeV-Aβ1-43/mIL-10 or the control vector.

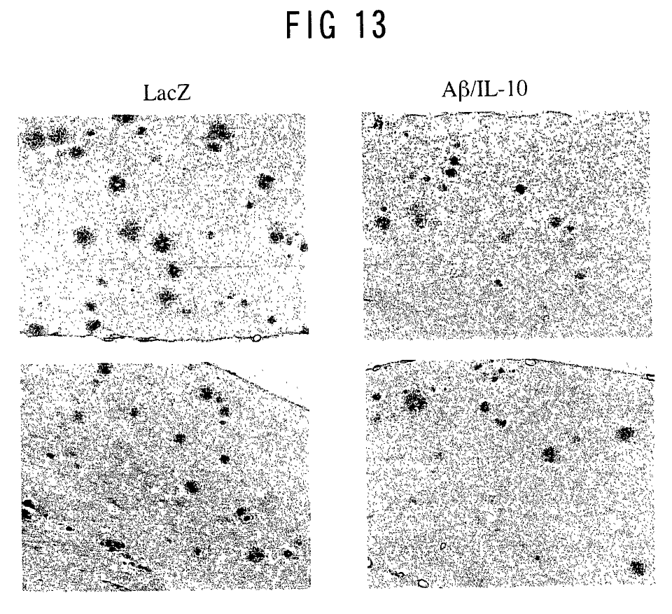

FIG. 14 is a photograph showing senile plaques in the hippocampus of APP transgenic mice 8 weeks after the intramuscular administration of SeV-Aβ1-43/mIL-10 or the control vector.

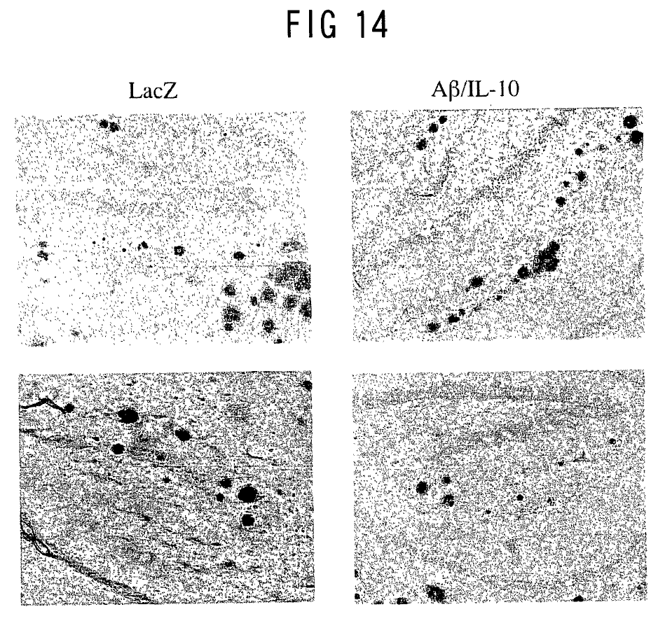

FIG. 15 is a graph showing the result of quantifying senile plaques in APP transgenic mice (frontal lobe, parietal lobe, and hippocampus) 8 weeks after the intramuscular administration of SeV-Aβ1-43/mIL-10 or the control vector.

FIG. 16 is a photograph showing the result of examining lymphocyte infiltration in the frontal lobe 8 weeks after the intramuscular administration of SeV-Aβ1-43/mIL-10or the control vector.

FIG. 17 is a set of graphs showing the amount of Aβ in the brain tissue 8 weeks after the intramuscular administration of SeV-Aβ1-43/mIL-10 or the control vector.

FIG. 18 is a graph showing the level of serum anti-Aβ antibody of normal mice after the intranasal administration of SeV-Aβ1-43/mIL-10.

FIG. 19 is a graph showing the level of serum anti-Aβ antibody of normal mice after the intranasal administration of Sendai virus therapeutic vectors encoding cytokines.

### BEST MODE FOR CARRYING OUT THE INVENTION

Herein below, the present invention will be specifically described using Examples, however, it is not to be construed as being limited thereto. All documents cited in the instant specification are incorporated by reference.

### Example 1

### Construction of a Minus Strand RNA Viral Genome cDNA Carrying an AβGene

### (1-1) Construction of NotI Fragment (Addition of a Transcription Signal of a Minus Strand RNA Virus)

PCR was performed using as a template, a gene (JP-A No. 2005-21149; SEQ ID NO: 1) encoding an Aβ peptide (SEQ ID NO: 28) which has been made into a secretory type by adding the secretion signal (amino acids 1 to 18) of an amyloid precursor protein (APP: Accession number AF282245) to a human Aβ peptide 1-43 (SEQ ID NO: 27), and two primers, phAbeta-NnotI (SEQ ID NO: 19) and phAbeta-CnotI (SEQ ID NO: 20). The resulting PCR products were digested with NotI, and then subcloned into pBluescript™ II KS (Stratagene) to construct a NotI fragment of the Aβ peptide gene comprising a Sendai virus transcription signal (SEQ ID NO: 21). Similarly, for mouse IL-10 (mIL10), PCR was performed using mIL10 cDNA (Accession number NM_{—}010548; SEQ ID NO: 2) as a template, and two primers pmIL10-N (SEQ ID NO: 22) and pmIL10-C (SEQ ID NO: 23). The resulting PCR products were digested with NotI, and then subcloned into pBluescript™ II KS (Stratagene) to construct a NotI fragment (SEQ ID NO: 24) of the mIL10 gene comprising a Sendai virus transcription signal.

SEQ ID NO:1

ATGCTGCCCGGTTTGGCACTGCTCCTGCTGGCCGCCTGGACGGCTCGGGC
GCTTGATGCAGAATTCCGACATGACTCAGGATATGAAGTTCATCATCAAA
AATTGGTGTTCTTTGCAGAAGATGTGGGTTCAAACAAAGGTGCAATCATT
GGACTCATGGTGGGCGGTGTTGTCATAGCGACTTAA

SEQ ID NO:19

ATTGCGGCCGCCAAGGTTCACTTATGCTGCCCGGTTTGGCACTGCTCCTG

SEQ ID NO:20

ATTGCGGCCGCGATGAACTTTCACCCTAAGTTTTTCTTACTACGGTTAAG
TCGCTATGACAACACCGCCCACCATGAGTCC

SEQ ID NO:21

gcggccgccaaggttcacttATGCTGCCCGGTTTGGCACTGCTCCTGCTG
GCCGCCTGGACGGCTCGGGCGCTTGATGCAGAATTCCGACATGACTCAGG
ATATGAAGTTCATCATCAAAAATTGGTGTTCTTTGCAGAAGATGTGGGTT
CAAACAAAGGTGCAATCATTGGACTCATGGTGGGCGGTGTTGTCATAGCG
ACTTAAccgtagtaagaaaaacttagggtgaaagttcatcgcggccgc

SEQ ID NO:2

ATGCCTGGCTCAGCACTGCTATGCTGCCTGCTCTTACTGACTGGCATGAG
GATCAGCAGGGGCCAGTACAGCCGGGAAGACAATAACTGCACCCACTTCC
CAGTCGGCCAGAGCCACATGCTCCTAGAGCTGCGGACTGCCTTCAGCCAG
GTGAAGACTTTCTTTCAAACAAAGGACCAGCTGGACAACATACTGCTAAC
CGACTCCTTAATGCAGGACTTTAAGGGTTACTTGGGTTGCCAAGCCTTAT
CGGAAATGATCCAGTTTTACCTGGTAGAAGTGATGCCCCAGGCAGAGAAG
CATGGCCCAGAAATCAAGGAGCATTTGAATTCCCTGGGTGAGAAGCTGAA
GACCCTCAGGATGCGGCTGAGGCGCTGTCATCGATTTCTCCCCTGTGAAA
ATAAGAGCAAGGCAGTGGAGCAGGTGAAGAGTGATTTTAATAAGCTCCAA
GACCAAGGTGTCTACAAGGCCATGAATGAATTTGACATCTTCATCAACTG
CATAGAAGCATACATGATGATCAAAATGAAAAGCTAA

SEQ ID NO:22

ACTTGCGGCCGCCAAAGTTCAATGCCTGGCTCAGCACTGCTATGCTGCCT
G

SEQ ID NO:23

ATCCGCGGCCGCGATGAACTTTCACCCTAAGTTTTTCTTACTACGGTTAG
CTTTTCATTTTGATCATCATGTATGCTTC

SEQ ID NO:24

gcggccgccaaagttcaATGCCTGGCTCAGCACTGCTATGCTGCCTGCTC
TTACTGACTGGCATGAGGATCAGCAGGGGCCAGTACAGCCGGGAAGACAA
TAACTGCACCCACTTCCCAGTCGGCCAGAGCCACATGCTCCTAGAGCTGC
GGACTGCCTTCAGCCAGGTGAAGACTTTCTTTCAAACAAAGGACCAGCTG
GACAACATACTGCTAACCGACTCCTTAATGCAGGACTTTAAGGGTTACTT
GGGTTTGCCAAGCCTTATCGGAAATGATCCAGTTTTACCTGGTAGAAGTG
ATGCCCCAGGCAGAGAAGCATGGCCCAGAAATCAAGGAGCATTTGAATTC
CCTGGGTGAGAAGCTGAAGACCCTCAGGATGCGGCTGAGGCGCTGTCATC
GATTTCTCCCCTGTGAAAATAAGAGCAAGGCAGTGGAGCAGGTGAAGAGT
GATTTTAATAAGCTCCAAGACCAAGGTGTCTACAAGGCCATGAATGAATT
TGACATCTTCATCAACTGCATAGAAGCATACATGATGATCAAAATGAAAA
GCTAAccgtagtaagaaaaacttagggtgaaagttcatcgcggccgc

(1-2) Insertion of mIL10 NotI Fragment into F Gene-Deleted Site

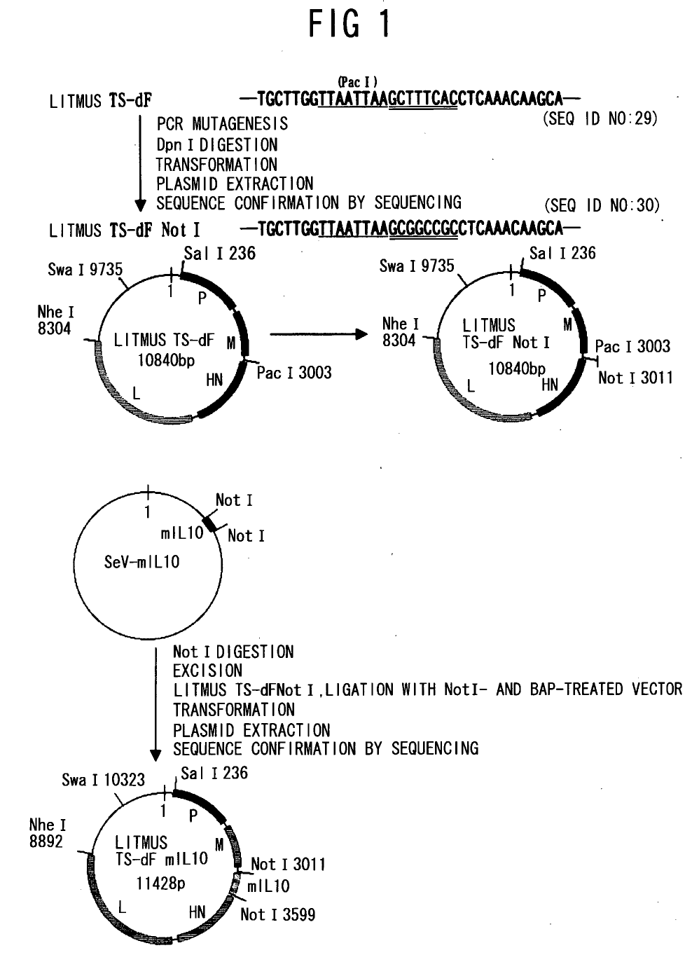

The cDNA (pSeV18+NotI/TSΔF) of an F gene-defective SeV vector (WO 2003/025570), to which amino acid mutations at 9 spots (M protein, G69E, T116A and A183S; HN protein, A262T, G264R and K461G; P protein, L₅₁₁F; L protein, N₁₁₉₇S and K1795E) had been introduced, was digested with SalI and NheI and subcloned into Litmus38 (New England Biolabs). Then, a mutation to insert a NotI cleavage site was introduced into the F gene-deleted site of the resulting Litmus TSΔF plasmid comprising M and HN genes. Specifically the insertion of the NotI cleavage site (FIG. 1, top drawing) was carried out by PCR using the Litmus TSΔF as a template and two primers F_lit3008_NotI (SEQ ID NO: 25) and R_lit3020_NotI (SEQ ID NO: 26). After the resulting Litmus TSΔF NotI was digested with NotI, the mIL10 gene NotI fragment was inserted (FIG. 1, bottom drawing).

| | |
|---|---|
| AAGCGGCCGCCTCAAACAAGCACAGATCATGGATG | SEQ ID NO:25 |
| AGGCGGCCGCTTAATTAACCAAGCACTCACAAGG | SEQ ID NO:26 |

(1-3) Construction of F Gene-Defective SeV cDNA Carrying Aβ Gene

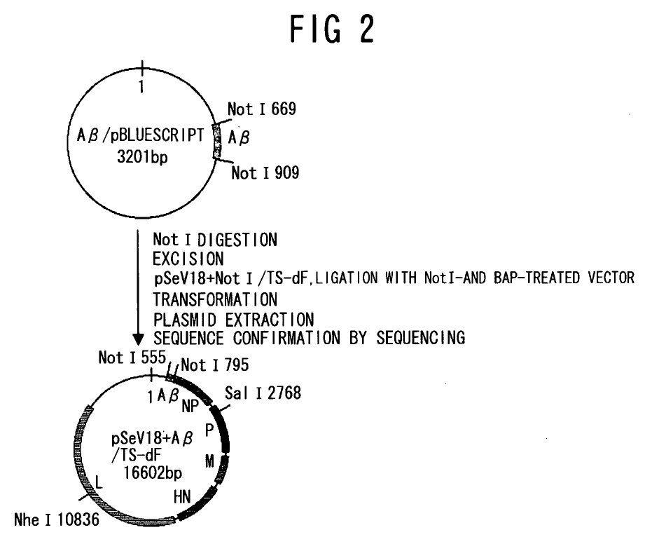

The Aβ peptide gene NotI fragment was inserted into the NotI site on pSeV18+NotI/TSΔF, which had been digested with NotI, to construct an F gene-defective SeV cDNA carrying the Aβ gene (pSeV18+Aβ/TSΔF) (FIG. 2).

(1-4) Construction of F Gene-Defective SeV Genome cDNA Carrying Both Aβ and IL-10 Genes

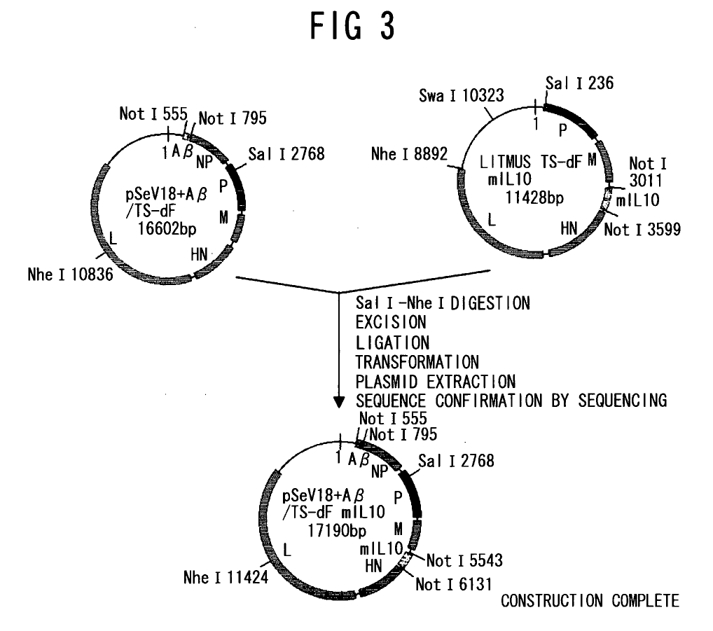

After digesting pSeV18+Aβ/TSΔF with SalI and NheI, a fragment not comprising M and HN genes was recovered. Meanwhile, Litmus TSΔF-mIL10 was digested with SalI and NheI to prepare a fragment comprising both M and HN genes. The two fragments were ligated to construct an F gene-defective SeV genome cDNA carrying both Aβ and IL-10 genes (pSeV18+Aβ/TSΔF-mIL10) (FIG. 3).

### Example 2

### Reconstruction and Amplification of Sendai Virus Vector

Viral reconstruction and amplification were carried out with the method reported by Li et al. (Li, H.-O. et al., J. Virology 74: 6564-6569, 2000; WO 00/70070) and its modified method (PCT/JP2005/00705). Since the viral vector is F gene-defective, a helper cell for supplying F protein was used. The helper cells were prepared using a Cre/loxP induced expression system. The system uses a plasmid pCALNdLw (Arai, T. et al., J. Virol. 72: 1115-1121, 1988), which is designed to induce expression of gene products by Cre DNA recombinase, and a recombinant adenovirus (AxCANCre) expressing Cre DNA recombinase to infect the transformant, which has been transformed with the pCALNdLw plasmid, according to the method of Saito et al. (Saito, I. et al., Nucl. Acid Res. 23: 3816-3821, 1995; Arai, T. et al., J. Virol. 72: 1115-1121, 1998).

The F gene-defective SeV vector carrying an Aβ gene (SeV18+Aβ/TSΔF) and the F gene-defective SeV vector carrying both Aft and IL-10 genes (SeV18+Aβ/TSΔF-mIL10) were prepared by the methods described above. The DNA sequence encoding the plus strand of SeV18+Aβ/TSΔF-mIL10 is shown in SEQ ID NO: 4, and the nucleotide sequence of the genome RNA (minus strand) is shown in SEQ ID NO: 5. A SeV vector carrying LacZ gene instead of the Aβ and IL-10 genes in the F gene-deleted site (SeV18+ LacZ /TSΔF; also referred to as “SeV LacZ”) was prepared as a control.

### Example 3

### Effect of Intranasally Administered SeV-Aβ1-43/mIL10 in an Alzheimer's Disease Animal Model

### (3-1) Animals and Method of Administration

Effect of SeV18+Aβ/TSΔF-mIL10 of the present invention (hereinafter also referred to as “SeV-Aβ1-43/mIL-10”) was tested using 24- to 25-month-old APP transgenic mice (Tg2576) (Hsiao, K., et al., Science 274:99-102, 1996). The mice were divided into two groups, the treatment group and control group, each consisting of four mice. SeV-Aβ1-43/mIL-10 (5×10⁶ CIU/head) was administered intranasally to the treatment group, and SeV LacZ (5×10⁶CIU/head) was administered intranasally to the control group.

### (3-2) Determination of Anti-Aβ Antibody in Serum

Blood was collected from the mice 4 and 8 weeks after the above-described treatment to determine the amount of anti-Aβ42 antibody present in serum. Aft 1-42 peptide (5 μg/mL) was adsorbed onto each well of a 96-well plate (Nunc, MaxiSorp). After blocking with 5% nonfat milk/TBS-T buffer, the mouse serum (500 times diluted) was added and detection was carried out using a peroxidase-labeled anti-mouse IgG antibody. Antibody titer was assessed with an absorbance measurement (O.D.450) using an ELISA reader.

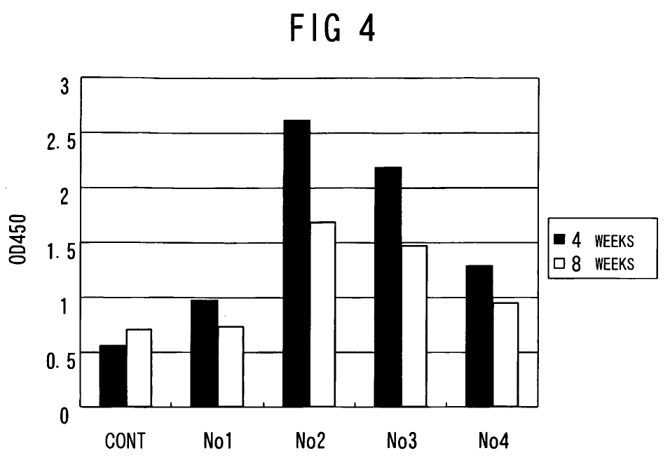

The result is shown in FIG. 4. The antibody titer was found to be markedly elevated in all subjects of the treatment group as compared with the control group.

(3-3) Senile Plaque-Eliminating Effect of SeV-Aβ1-43/mIL-10

The mice intranasally administered the above-described Sendai virus vector were killed 8 weeks after the administration (26 or 27 months old), and the brain tissue sections of the cortex of frontal lobe, parietal lobe, and hippocampus were prepared. The frozen sections of the above-mentioned tissues were treated as described below. To detect Aβ proteins or senile plaques, the tissue sections were treated with 70% formic acid and endogenous peroxidase was inactivated with 5% H₂O₂. Then the tissue sections were reacted with a rabbit anti-pan-Aβ antibody (1000 times diluted), and a peroxidase-labeled secondary antibody was added, followed by DAB staining. The stained sections were observed using a 3 CCD camera connected to a microscope, and the area of Aβ accumulation was measured in each region and the area fraction was calculated.

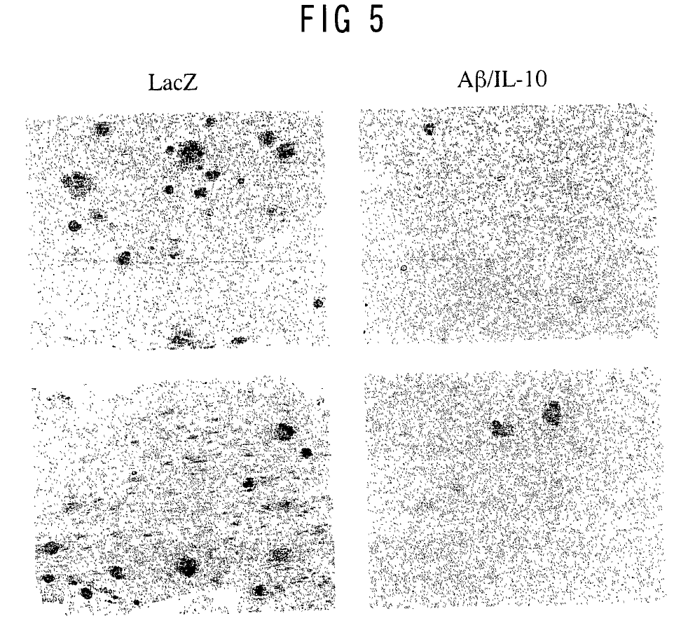

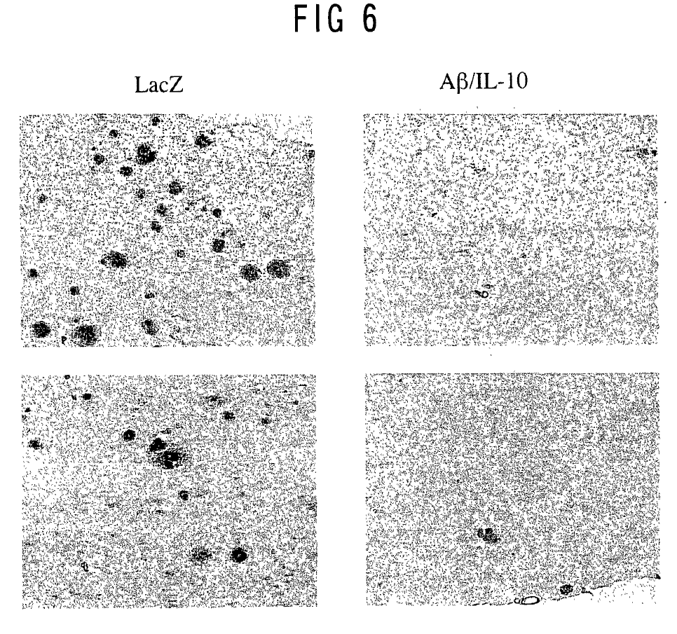

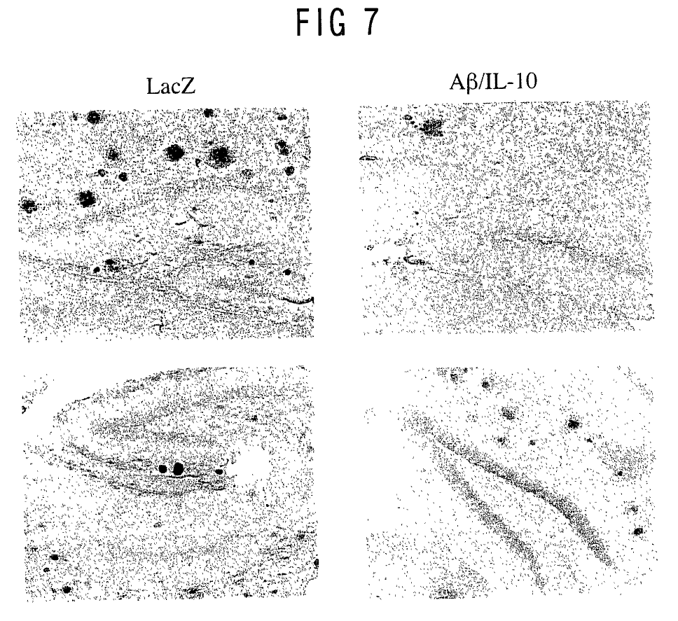

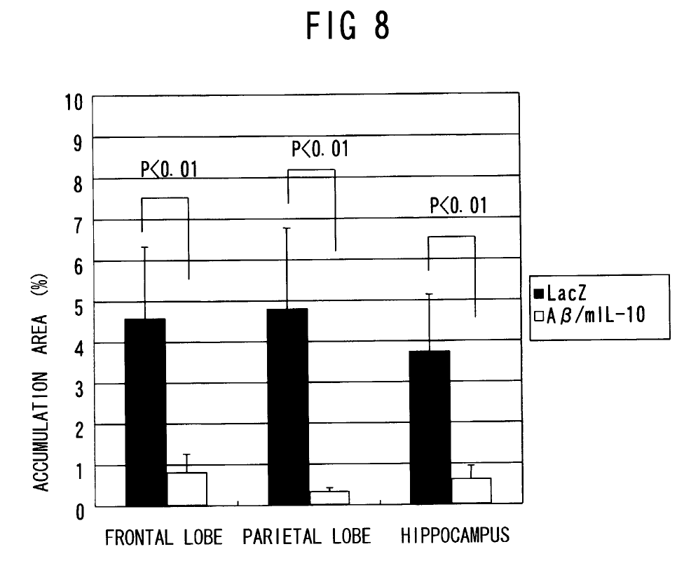

The results of section staining are shown in FIG. 5 (frontal lobe), FIG. 6 (parietal lobe), and FIG. 7 (hippocampus). The area ratio of Aβ deposition is shown in FIG. 8. It was confirmed that SeV-Aβ1-43/mIL-10 administration markedly reduced senile plaques to 10 to 20% of the control in all of the frontal and parietal lobes, and hippocampus.

### (3-4) Safety Evaluation of SeV-Aβ1-43/mIL Administration

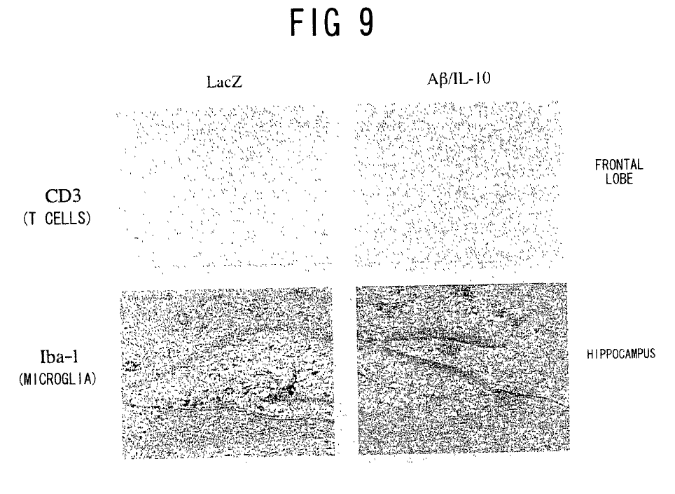

Frozen tissue sections of the frontal lobe and hippocampus were prepared from the treatment group and the control group 8 weeks after the administration (26- to 27-months-old) using the same procedure described above in (3-3). The frozen sections were stained with an ABC method using an anti-CD3 antibody (T cells) and an anti-Iba-1 antibody (microglia) to assess whether lymphocyte infiltration in the central nervous system and microglial alteration took place. As shown in FIG. 9, infiltration of T lymphocytes (cells positive for CD3, which is a pan T cell marker) was not observed in both the frontal lobe and hippocampus in either the treatment group or the control. Thus, the possibility that inflammation of the central nervous system may be caused by using the present vectors for treatment was suggested to be very low. Furthermore, the quantity of cells positive for IBA-1, a microglial marker, was comparable between the control group and the treatment group, confirming that the treatment did not result in an extreme activation (accumulation) in the treatment group.

### (3-5) Determination of Aβ in Brain Tissues

The cerebrum and cerebellum of the mice were dissected along the fissura mediana, and the hemispheres were snap frozen and stored at −80° C. The brain hemisphere was homogenized in 1 ml of TBS solution, and centrifuged at 100,000 g for one hour using a benchtop ultracentrifuge. The soluble fraction (TBS fraction) was stored. The insoluble fraction was dissolved in 2% SDS and homogenized, and then centrifuged again at 100,000 g for one hour. The resulting soluble fraction (2% SDS fraction) was stored. The insoluble fraction was dissolved in 70% formic acid and homogenized, and then centrifuged again at 100,000 g for one hour. The resulting soluble fraction (formic acid fraction) was stored. Aβ40 and 42 in the brain tissues were determined using a BioSource ELISA kit. The TBS fraction was diluted 4 times; the 2% SDS fraction was diluted 400 to 2000 times; and the formic acid fraction was diluted 1000 times with 1 M Tris solution. Then, the diluted fractions were further diluted (2 to 10 times) with an ELISA dilution solution for determination.

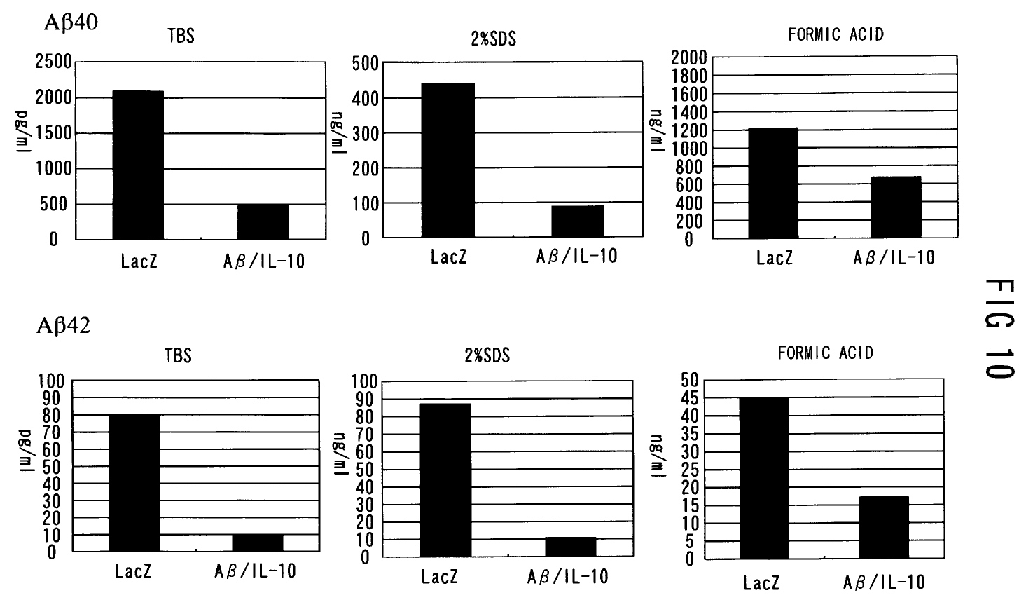

As shown in FIG. 10, it was proven that SeV-Aβ1-43/mIL-10 administration markedly reduced the level of Aβ in the brain tissues in all of the SDS, formic acid, and TBS fractions.

### Example 4

### Effect of Intramuscularly Administered SeV-Aβ1-43/mIL-10 in an Alzheimer's Disease Animal Model

### (4-1) Animals and Method of Administration

Except for the administration route, all the procedures were the same as that used in the intranasal administration described above. SeV-Aβ1-43/mIL-10 vector was administered to the hind thigh muscle at a dose of 5×10⁶ CIU.

### (4-2) Determination of Anti-Aβ Antibody in Serum

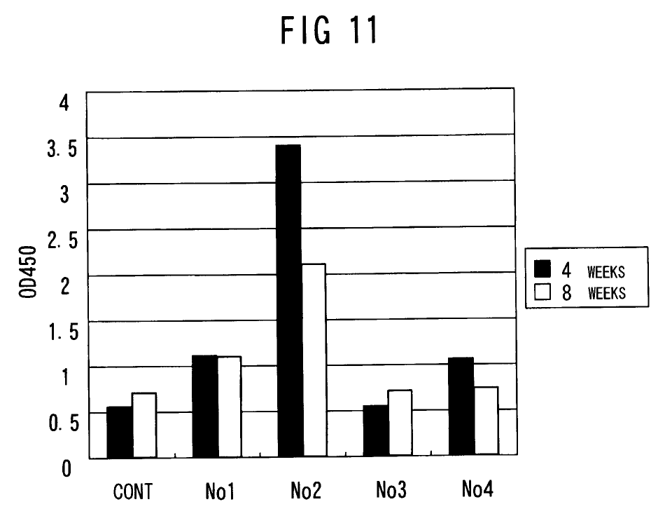

The amount of anti-Aβ antibody in the serum of mice that had been intramuscularly administered SeV-Aβ1-43/mIL-10 vector was determined by the same method as used for the intranasal administration described above. The result is shown in FIG. 11.

### (4-3) Senile Plaque-Eliminating Effect of SeV-Aβ1-43/mIL

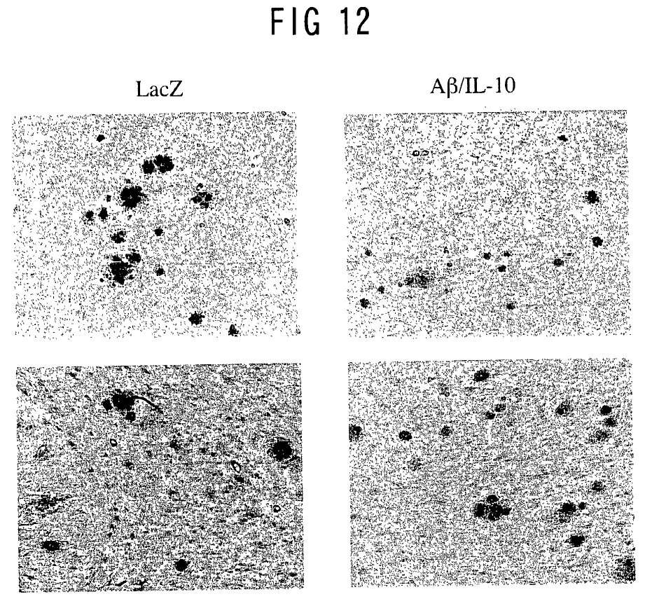

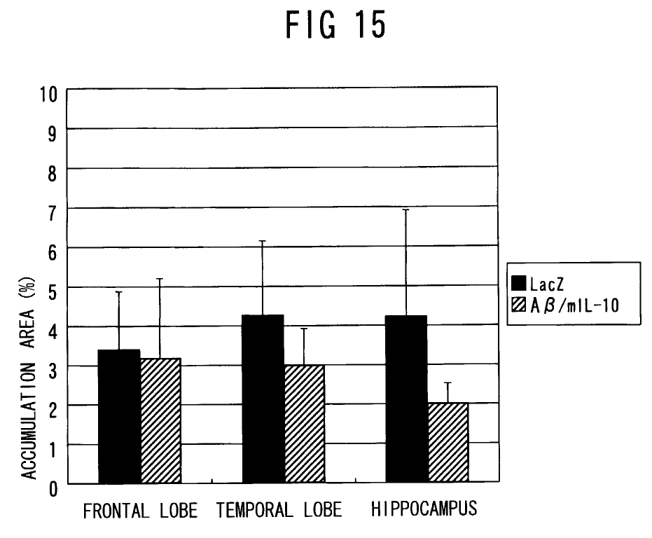

The senile plaque-eliminating effect in the frontal lobe, parietal lobe, and hippocampus after intramuscular administration of SeV-Aβ1-43/mIL-10 vector was assessed by the same method as used for the intranasal administration described above. The results are shown in FIGS. 12 (frontal lobe), 13 (parietal lobe), and 14 (hippocampus). The area ratio of Aβ deposition is shown in FIG. 15. Each region showed a certain degree of Aβ deposition reduced.

(4-4) Safety Evaluation of SeV-Aβ1-43/mIL-10 Administration

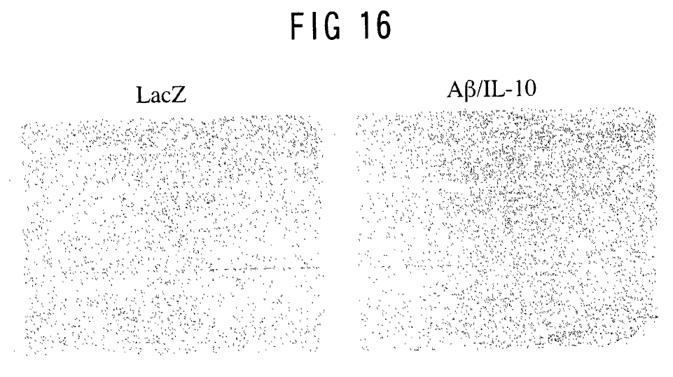

The presence of lymphocyte infiltration in the central nervous system after intramuscular administration of the SeV-Aβ1-43/mIL-10 vector was assessed by the same method as used for the intranasal administration described above. As shown in FIG. 16, infiltration of T lymphocytes in the frontal lobe was not observed in either the control group or the treatment group.

### (4-5) Determination of Aβ in Brain Tissues

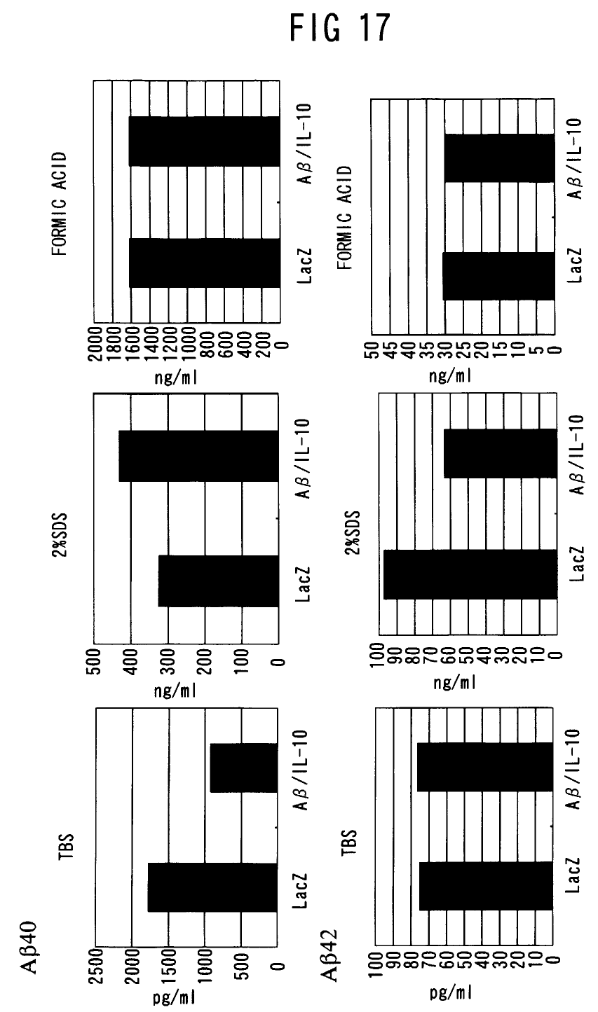

Aβ40 and Aβ42 in brain tissues after intramuscular administration of the SeV-Aβ1-43/mIL-10 vector were determined by the same method as used for the intranasal administration described above. The result is shown in FIG. 17. It was confirmed that the level of Aβ decreased in some soluble fractions.

### Example 5

### Elevation of the Antibody Titer by Intranasal Administration of SeV-Aβ1-43/hIL-10 in Normal Mice

### (5-1) Animals and Method of Administration

Effect of SeV18+Aβ1-43/TSΔF-hIL10 (hereinafter also referred to as “SeV-Aβ1-43/hIL-10”) of the present invention was tested using 6-week-old C57BL/6N mice. The mice were divided into three groups, each consisting of six mice. SeV-Aβ1-43/hIL-10 (group 1: 5×10⁵ CIU/head, group 2: 5×10⁶, group 3: 5×10⁷ CIU/head) was administered intranasally to each mouse.

### (5-2) Determination of Anti-Aβ Antibody in Plasma

Blood was collected from the mice before and 2, 4 and 8 weeks after the administration to determine the amount of anti-Aβ42 antibody present in plasma. Aβ1-42 peptide (4 μg/mL) was adsorbed onto each well of a 96-well plate (Nunc, MaxiSorp). After blocking with 1% BSA/2% goat serum/TBS-T buffer, the mouse plasma (300 times diluted) were added and detection was carried out using a peroxidase-labeled anti-mouse IgG antibody. Antibody titer was assessed with an absorbance measurement (O.D.450) using an ELISA reader.

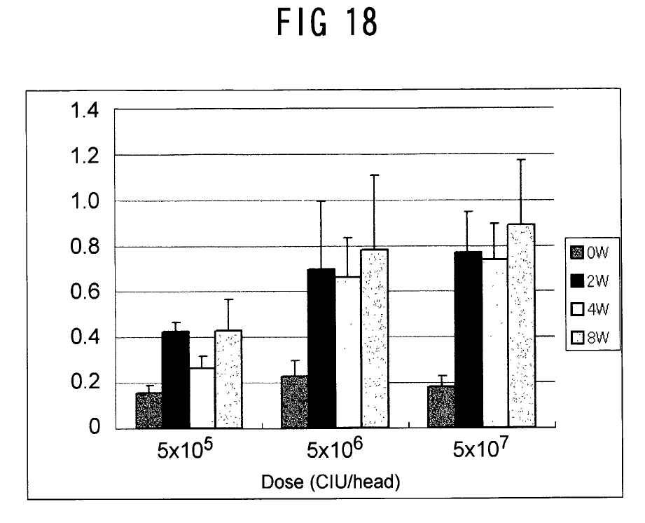

The result is shown in FIG. 18. The amount of anti-Aβ antibody was increased during the time course in all the three groups. While the amount of the antibody was increased by the vector administration in dose dependent manner, significant difference of the amount of the antibody was not observed between group 2 (5×10⁶ CIU/head) and group 3 (5×10⁷ CIU/head). The result shows that the induction of anti-Aβ antibody by the administration of the reagent of the invention (SeV-Aβ1-43/hIL-10) is not specific to Alzheimer's disease model mice (APP Tg mice), but is sufficiently achieved in normal mice.

### Example 6

### Effects of Cytokine-Encoding Therapeutic Vectors for Alzheimer's Disease in Intranasal Administration to Normal Mice

### (6-1) Animals and Method of Administration

Gene encoding several cytokines were inserted into the therapeutic vectors for Alzheimer's disease described above. Effect of these Sendai virus vectors were tested using 6-week-old C57BL/6N mice. The mice were divided into three groups, each consisting of six mice. The Sendai virus vector (carrying a gene encoding Aβ peptide as well as a gene encoding mouse IL-4, IL-5 or IL-6; referred to as SeV-Aβ1-42/mIL-4, SeV-Aβ1-42/mIL-5 or SeV-Aβ1-42/mIL-6, respectively) was administered intranasally to each mouse (5×10⁶ CIU/10 μl).

### (6-2) Determination of Anti-Aβ Antibody in Plasma

Blood was collected from the mice before and 1 and 2 weeks after the administration to determine the amount of anti-Aβ antibody present in plasma. Aβ1-42 peptide (4 μg/mL) was adsorbed onto each well of a 96-well plate (Nunc, MaxiSorp). After blocking with 1% BSA/2% goat serum/TBS-T buffer, the mouse plasma (300 times diluted) were added and detection was carried out using a peroxidase labeled anti-mouse IgG antibody. Antibody titer was assessed with an absorbance measurement (O.D.450) using an ELISA reader.

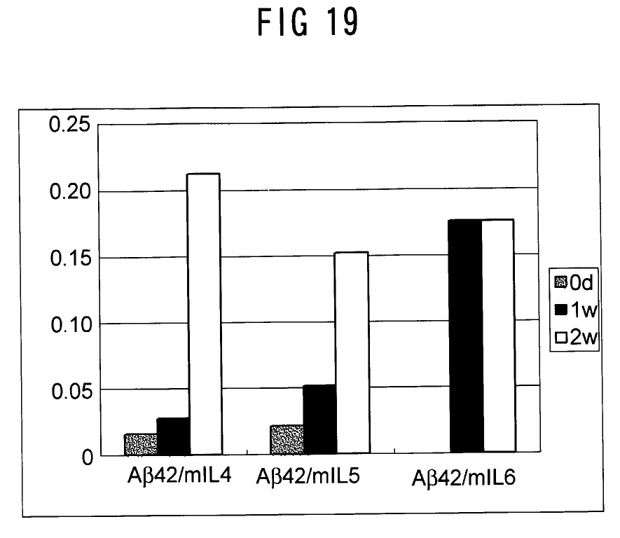

The result is shown in FIG. 19. The amount of anti-Aβ antibody was increased during the time course in the all three groups. The result shows that IL-4, IL-5, and IL-6 are effective for inducing anti-Aβ antibody, and the formulation comprising the Sendai virus vectors carrying both nucleic acids encoding an Aβ antigen and one or more cytokines selected from IL-4, IL-5, and IL-6 are preferable for inducing anti-Aβ antibody in treatment for Alzheimer's disease.

### INDUSTRIAL APPLICABILITY

The present invention provides novel minus strand RNA viral vectors that can effectively reduce the level of amyloid β deposition. The vectors of the present invention can be used to treat Alzheimer's disease.

1. A paramvxoviral vector comprising (i) an amyloid β-encoding nucleic acid and (ii) a nucleic acid encoding a Th2 cytokine or a partial peptide thereof.

2. (canceled)

3. The paramvxoviral vector of claim 1, wherein the Th2 cytokine or a partial peptide thereof is IL-10 or a partial peptide thereof.

4. (canceled)

5. The vector of claim 1, wherein the paramyxoviral vector is a Sendai virus vector.

6. The vector of claim 1, wherein the amyloid β is Aβ43 or a partial peptide thereof.

7. The vector of claim 1, wherein the amyloid β is derived from human.

8-11. (canceled)

12. A composition which comprises the vector of claim 1 and a pharmaceutically acceptable carrier.

13-21. (canceled)

22. A method for reducing deposition of amyloid β, wherein the method comprises a step of administering the vector of claim 1, or a composition comprising the vector.

23. A method for treating or preventing Alzheimer's disease, wherein the method comprises a step of administering the vector of claim 1, or a composition comprising the vector.

24. The method of claim 22 wherein the administration is intranasal administration.

25. The method of claim 22, wherein the administration is intramuscular injection.

26-28. (canceled)

29. The method of claim 23 wherein the administration is intranasal administration.

30. The method of claim 23, wherein the administration is intramuscular injection.
